(19) United States
(12) Patent Application Publication
(10) Pub. No.: US 2023/0390424 A1
(43) Pub. Date: Dec. 7, 2023

(54) TROPHOBLAST GLYCOPROTEIN RADIOIMMUNOTHERAPY FOR THE TREATMENT OF SOLID CANCERS

(71) Applicant: ACTINIUM PHARMACEUTICALS, INC., New York, NY (US)

(72) Inventors: Dale L. LUDWIG, Rockaway, NJ (US); Eileen GEOGHEGAN, Mamaroneck, NY (US)

(21) Appl. No.: 18/025,849

(22) PCT Filed: Sep. 13, 2021
(86) PCT No.: PCT/US2021/050009

§ 371 Date: Mar. 10, 2023

Related U.S. Application Data
(60) Provisional application No. 63/077,297, filed on Sep. 11, 2020.

(51) Int. Cl. A61K 51/10; A61K 39/395; A61K 31/502; A61K 31/454; A61K 31/55; A61K 31/5025; A61P 35/00

(52) U.S. Cl. CPC A61K 51/1096; A61K 39/3955; A61K 31/502; A61K 31/454; A61K 31/55; A61K 31/5025; A61P 35/00; A61K 2039/505

(57) ABSTRACT

Compositions and methods for treating a solid cancer in a subject by administering an effective amount of a radioisotope labeled trophoblast glycoprotein (5T4)-targeting agent are provided. The 5T4-targeting agent may be an antibody against 5T4 labeled with ²²⁵Ac, ¹⁷⁷Lu, ¹³¹I, ⁹⁰Y, ²¹³Bi, ²¹¹At, ²¹²Bi, ²²⁷Th, or ²¹²Pb. The solid cancer may be a 5T4-positive tumor. The effective amount of the radiolabeled 5T4-targeting agent may be a maximum tolerate dose administered in a single bolus or in fractionated doses that together equal the maximum tolerated dose. The methods may further include administration of additional agents, such as chemotherapeutic agents, immune checkpoint therapies, and/or DNA damage response inhibitors.

### CROSS-REFERENCE TO RELATED APPLICATIONS

This application claims priority to U.S. provisional application Ser. No. 63/077,297 filed Sep. 11, 2020 which is hereby incorporated by reference in its entirety.

### SEQUENCE LISTING

The instant application contains a Sequence Listing which has been submitted electronically in ASCII format and is hereby incorporated by reference in its entirety. Said ASCII copy, created on Sep. 12, 2021, is named ATNM-006PCT-SL.txt and is 102,978 bytes in size.

### FIELD OF THE INVENTION

The present invention relates to relates the field of radiopharmaceuticals.

### BACKGROUND OF THE INVENTION

Trophoblast glycoprotein (TBPG), also known as 5T4, is a glycoprotein that is categorized as an oncofetal antigen, meaning it is expressed on cells during fetal developmental stages but is not expressed in adult tissues except on tumors (Southall, 1990). 5T4 is expressed widely across many different tumor types, including lung, breast, head and neck, colorectal, bladder, ovarian, pancreatic, and many others (Stern, 2017). Additional characteristics that make it amenable for targeting with an armed antibody include a high rate of internalization, expression on the tumor periphery, and expression on cancer stem cells (Harper, 2017; Boghaert, 2008; Sapra, 2013).

Several attempts have been made to develop therapeutics against tumors through expression, including antibodies (Harper, 2017; Shapiro, 2017), vaccines (Harrop, 2013), and cellular therapies (Owens, 2018). While an unlabeled 5T4-targeting antibody is not an effective therapeutic (Boghaert, 2008), armed antibodies such as antibody drug-conjugates (ADC) with toxins such as calicheamicin (Boghaert, 2008), auristatin (Sapra, 2018; Shi, 2019; Wang, 2018), tubulysin (Harper, 2017), and pyrrolobenzodiazepine (Harper, 2017; Kerk, 2017) have been developed and tested preclinically. The auristatin based ADC developed by Pfizer was tested clinically, with no objective responses reported and toxicity related to the auristatin conjugate observed (Shapiro, 2017). A 5T4-targeted antibody has also been conjugated with the Staphylococcus enterotoxin A (SEA) super antigen and has been evaluated preclinically and clinically, with only limited clinical efficacy observed (Cheng 2004; Shaw 2007; Borghaei 2009).

Altogether, this body of evidence suggests that while targeting the 5T4 antigen may be safe, the previously tested 5T4-targeting modalities are inadequate to control tumor growth. Accordingly, an object of the presently disclosed invention is to provide 5T4 targeting agents that are effective in treating 5T4-positive proliferative disorders and cancers and in killing cancer cells.

### SUMMARY OF THE INVENTION

The presently disclosed invention provides a novel radio-conjugate approach for targets. More specifically, the present invention is related to a 5T4 targeting agent, such as a monoclonal antibody, peptide, or small molecule that targets 5T4, labeled with a radioisotope, and methods of diagnosing and treating 5T4-positive cancers using the radiolabeled 5T4 targeting agent and of killing 5T4-positive cells, such as cancer cells, generally.

According to certain aspects of the present invention, the 5T4 targeting agent useful for diagnostics purposes may be an anti-5T4 antibody, peptide, or small molecule including a radioisotope, such as ¹¹¹In, ⁶⁸Ga, or ⁸⁹Zr.

According to certain other aspects, the 5T4 targeting agent useful for therapeutic interventions may be an anti-5T4 antibody, peptide, or small molecule including a radioisotope, such as: ¹³¹I, ¹²⁵I, ¹²³I, ⁹⁰Y, ¹⁷⁷Lu, ¹⁸⁶Re, ¹⁸⁸Re, ⁸⁹Sr, ¹⁵³Sm, ³²P, ²²⁵Ac, ²¹³Bi, ²¹³Po, ²¹¹At, ²¹²Bi, ²¹³Bi, ²²³Ra, ²²⁷Th, ¹⁴⁹Tb, ¹³⁷Cs, ²¹²Pb, or combinations thereof. According to certain preferred aspects, the 5T4 targeting agent may include ¹³¹I, ⁹⁰Y, ¹⁷⁷Lu, ²²⁵Ac, ²¹³Bi, ²¹¹At, ²²⁷Th, or ²¹²Pb.

According to certain aspects, the 5T4-positive cancer may be a solid tumor. According to other aspects, the 5T4-positive cancer is a hematological cancer such as acute lymphoblastic leukemia (ALL) such as Philadelphia chromosome-positive ALL.

Therapeutic methods of the presently disclosed invention generally include administering to a patient an effective amount of the 5T4 targeting agent. According to certain aspects, the effective amount of the 5T4 targeting agent may be a maximum tolerated dose (MTD) or may be a fractioned dose wherein the total amount of radiation administered in the fractioned doses is the MTD.

According to certain aspects, the 5T4 targeting agent includes a radiolabeled fraction and a non-radiolabeled fraction. As such, an effective amount of the 5T4 targeting agent may, for example, include a total dose of less than 100 mg, such as from 5 mg to 60 mg, or 5 mg to 45 mg. According to certain aspects, the total dose may be from 0.001 mg/kg to 3 mg/kg body weight of the subject, such as from 0.005 mg/kg to 2 mg/kg body weight of the subject. According to certain aspects, the total dose may be less than 2 mg/kg, or less than 1 mg/kg, less than 0.5 mg/kg, or even less than 0.1 mg/kg. A portion of the total dose is radiolabeled (i.e., radio-conjugated) as indicated, wherein the effective amount of the radiolabeled 5T4 targeting agent may depend on the specific radioisotope selected. Preferred radioisotopes for therapeutic interventions include ²²⁵Ac, ¹⁷⁷Lu, ¹³¹I, ⁹⁰Y, ²¹³Bi, ²¹¹At, ²²⁷Th, or ²¹²Pb. Thus, the 5T4 targeting agent may include a radiolabeled fraction and an unlabeled fraction.

According to certain aspects, an effective amount of an 5T4 targeting agent, such as an ²²⁵Ac-anti-5T4 antibody, peptide, or small molecule, may include a dose of 0.1 to 20 μCi/kg body weight of the subject, such as 0.1 to 10 μCi/kg or 0.1 to 5 μCi/kg body weight of the subject, or 0.5 to 20 μCi/kg or 1 to 10 μCi/kg body weight of the subject.

According to certain aspects, the effective amount of the 5T4 targeting agent, such as an ²²⁵Ac-anti-5T4 antibody, peptide, or small molecule may depend on the configuration of the targeting agent, i.e., full length antibody or antibody fragment (e.g., minibody, nanobody, etc). For example, when the 5T4 targeting agent may include an ²²⁵Ac-anti-5T4 antibody that is a full-length antibody, and the dose may be below 5 μCi/kg body weight of the subject, such as 0.1 to 5 μCi/kg body weight of the subject. In an alternative example, when the 5T4 targeting agent includes an ²²⁵Ac-anti-5T4 antibody that is a fragment, the dose may be greater than 5 μCi/kg body weight of the subject, such as 5 to 20 μCi/kg body weight of the subject.

Exemplary 5T4-specific antibodies that may be used include but are not limited to the following molecules or the antibody components of the following: Naptumomab estafenatox developed by Active Biotech AB (Lund, Sweden); MEDI0641, described in Harper (2017) and developed by Medimmune/AstraZeneca; ALG.APV-527, developed by Aptevo Therapeutics/Alligator Bioscience; Tb535, developed by Biotecnol/Chiome Bioscience; H6-DM5, described in Wang (2018) and developed by Guangdong Zhongsheng Pharmaceuticals; and ZV0508, described in Shi (2019) and developed by Zova Biotherapeutics.

According to certain aspects, the 5T4 targeting agent may be administered according to a dosing schedule selected from the group consisting of one dose every 7, 10, 12, 14, 20, 24, 28, 35, and 42 days throughout a treatment period, wherein the treatment period includes at least two doses.

According to certain aspects, the 5T4 targeting agent may be administered according to a dose schedule that includes 2 doses, such as on days 1 and 5, 6, 7, 8, 9, or 10 of a treatment period, or days 1 and 8 of a treatment period.

According to certain aspects, the 5T4 targeting agent may be administered as a single bolus or infusion in a single subject specific dose.

According to certain aspects, the methods may further include administration of one or more further therapeutic agents, such as a chemotherapeutic agent, an anti-inflammatory agent, an immunosuppressive agent, an immunomodulatory agent, an antimyeloma agent, a cytokine, or a combination thereof.

According to certain aspects, the methods may further include administration of one or more immune checkpoint therapies. Exemplary immune checkpoint therapies that may be used include an antibody against CTLA-4, PD-1, TIM-3, VISTA, BTLA, LAG-3, TIGIT, CD28, OX40, GITR, CD137, CD40, CD40L, CD27, HVEM, PD-L1, PD-L2, PD-L3, PD-L4, CD80, CD86, CD137-L, GITR-L, CD226, B7-H3, B7-H4, BTLA, TIGIT, GALS, KIR, 2B4, CD160, CGEN-15049, or any combination thereof. According to certain aspects, the immune checkpoint therapy may include an antibody against an immune checkpoint protein selected from the group consisting of an antibody against PD-1, PD-L1, PD-L2, CTLA-4, CD137, and a combination thereof.

According to certain aspects, the immune checkpoint therapy may be provided in a subject effective amount including a dose of 0.1 mg/kg to 50 mg/kg of the patient's body weight, such as 0.1-5 mg/kg, or 5-30 mg/kg.

According to certain aspects, the methods may further include administration of one or more DNA damage response inhibitors (DDRi). An exemplary DDRi that may be used includes at least one or more antibodies against poly(ADP-ribose) polymerase (i.e., PARPi). According to certain aspects, the PARPi may be selected from the group consisting of olaparib, niraparib, rucaparib, talazoparib, and a combination thereof. According to certain aspects, the PARPi may be provided in a subject effective amount including 0.1 mg/day-1200 mg/day, such as 0.100 mg/day-600 mg/day, or 0.25 mg/day-1 mg/day. Exemplary subject effective amounts that may be used include 0.1 mg, 0.25 mg, 0.5 mg, 0.75 mg, 1.0 mg, 100 mg, 200 mg, 300 mg, 400 mg, 500 mg, 600 mg, 700 mg, 750 mg, 800 mg, 900 mg, and 1000 mg, taken orally in one or two doses per day.

Another exemplary DDRi that may be used includes an inhibitor of Ataxia telangiectasia mutated (ATM), Ataxia telangiectasia mutated and Rad-3 related (ATR), or Wee1. Exemplary inhibitors of ATM include KU-55933, KU-59403, wortmannin, CP466722, and KU-60019. Exemplary inhibitors of ATR include at least Schisandrin B, NU6027, NVP-BEA235, VE-821, VE-822, AZ20, and AZD6738. Exemplary inhibitors of Wee1 that may be used include AZD-1775 (i.e., adavosertib).

According to certain aspects, the methods may further include administration of a combination of further therapeutic agents. Exemplary combinations that may be used include at least one or more DDRi and/or one or more immune checkpoint therapies and/or one or more CD47 blockades.

According to certain aspects, the 5T4 targeting agent and the one or more further therapeutic agents may be administered simultaneously or sequentially. When more than one additional therapeutic agent is administered, the agents may be administered simultaneously or sequentially.

According to certain aspects of the present invention, the 5T4 targeting agent may be a portion of a multi-specific antibody. Thus, the methods may include administering to the subject an effective amount of a multi-specific antibody, wherein the multi-specific antibody includes: a first target recognition component which specifically binds to an epitope of 5T4, and a second target recognition component which specifically binds to a different epitope of 5T4 than the first target recognition component, or an epitope of a different antigen.

Additional features, advantages, and aspects of the invention may be set forth or apparent from consideration of the following detailed description, drawings if any, and claims. Moreover, it is to be understood that both the foregoing summary of the invention and the following detailed description are exemplary and intended to provide further explanation without limiting the scope of the invention as claimed.

### DETAILED DESCRIPTION

In one aspect, the presently disclosed invention further provides methods for the treatment of 5T4-positive cancers, i.e., cancers having cells expressing 5T4. These methods generally include administering to the patient an effective amount of a radiolabeled 5T4 targeting agent, such as a radiolabeled antibody, peptide, or small molecule targeted to 5T4, alone or in combination with one or more additional therapeutic agents. The additional therapeutic agents include at least one or more immune checkpoint therapies and/or one or more inhibitors of a component of the DNA damage response pathway (i.e., a DNA damage response inhibitor, DDRi, such as one or more antibodies against poly(ADP-ribose) polymerase, i.e., PARPi). Additional therapeutic agents may further include a chemotherapeutic agent, a small molecule cancer therapeutic, an anti-inflammatory agent, an immunosuppressive agent, an immunomodulatory agent, an antimyeloma agent, a cytokine, or a combination thereof.

A related aspect of the invention provides a method for killing 5T4-expressing cells, such as 5T4-expressing cancer cells or 5T4-expressing precancerous cells that involves contacting such cells with a radiolabeled 5T4 targeting agent such as a radiolabeled antibody, peptide, or small molecule targeted to 5T4 to deliver radiation to the cells, alone or in combination with one or more additional agents such as chemotherapeutic agents and small molecule cancer therapeutics.

The presently disclosed invention also provides methods for detecting trophoblast glycoprotein positive (5T4-positive) cancer/tumor cells in a mammalian subject, and methods of diagnosing 5T4-positive cancers in a mammalian subject, which may, for example, be employed prior to treating a subject for a 5T4-expressing cancer using a radiolabeled 5T4 targeting agent.

Prior to setting forth the invention in greater detail, it may be helpful to an understanding thereof to set forth definitions of certain terms to be used hereinafter.

### Definitions and Abbreviations

The singular forms “a,” “an,” “the” and the like include plural referents unless the context clearly dictates otherwise. Thus, for example, reference to “an” antibody includes both a single antibody and a plurality of different antibodies.

The words “comprising” and forms of the word “comprising” as well as the word “including” and forms of the word “including” as used in this description and in the claims, do not limit the inclusion of elements beyond what is referred to. Additionally, although throughout the present disclosure various aspects or elements thereof are described in terms of “including” or “comprising,” corresponding aspects or elements thereof described in terms of “consisting essentially of” or “consisting of” are similarly disclosed. For example, while certain aspects of the invention have been described in terms of a method “including” or “comprising” administering a radiolabeled targeting agent, corresponding methods instead reciting “consisting essentially of” or “consisting of” administering the radiolabeled target are also within the scope of said aspects and disclosed by this disclosure. In this context, “consisting essentially of” provides a scope permitting the presence of elements that do not materially affect the basic and novel or functional characteristic(s) of what is referred to, while the term “consisting of” excludes any element beyond what is referred to.

The term “about” when used in this disclosure in connection with a numerical designation or value, e.g., in describing temperature, time, amount, and concentration, including in the description of a range, indicates a variance of ±10% and, within that larger variance, variances of ±5% or ±1% of the numerical designation or value.

As used herein, “administer”, with respect to a targeting agent such as an antibody, antibody fragment, Fab fragment, or aptamer, means to deliver the agent to a subject's body via any known method suitable for antibody delivery. Specific modes of administration include, without limitation, intravenous, transdermal, subcutaneous, intraperitoneal, intrathecal and intra-tumoral administration. Exemplary administration methods that may be used for antibodies may be as substantially described in International Pub. No. WO 2016/187514, incorporated by reference herein.

In addition, in this invention, antibodies can be formulated using one or more routinely used pharmaceutically acceptable carriers. Such carriers are well known to those skilled in the art. For example, injectable drug delivery systems include solutions, suspensions, gels, microspheres and polymeric injectables, and can include excipients such as solubility-altering agents (e.g., ethanol, propylene glycol and sucrose) and polymers (e.g., polycaprylactones and PLGA's).

As used herein, the term “antibody” includes, without limitation, (a) an immunoglobulin molecule including two heavy chains and two light chains and which recognizes an antigen; (b) polyclonal and monoclonal immunoglobulin molecules; (c) monovalent and divalent fragments thereof, such as Fab, di-Fab, scFvs, diabodies, minibodies, and nanobodies (sdAb); (d) naturally occurring and non-naturally occurring, such as wholly synthetic antibodies, IgG-Fc-silent, and chimeric; and (e) bi-specific forms thereof. Immunoglobulin molecules may derive from any of the commonly known classes, including but not limited to IgA, secretory IgA, IgG and IgM. IgG subclasses are also well known to those in the art and include, but are not limited to, human IgG1, IgG2, IgG3 and IgG4. The N-terminus of each chain defines a “variable region” of about 100 to 110 or more amino acids primarily responsible for antigen recognition. The terms variable light chain (VL) and variable heavy chain (VH) refer to these regions of light and heavy chains respectively. Antibodies may be human, humanized or nonhuman. When a specific aspect of the presently disclosed invention refers to or recites an “antibody,” it is envisioned as referring to any of the full-length antibodies or fragments thereof disclosed herein, unless explicitly denoted otherwise.

A “humanized” antibody refers to an antibody in which some, most or all amino acids outside the CDR domains of a non-human antibody are replaced with corresponding amino acids derived from human immunoglobulins. In one embodiment of a humanized form of an antibody, some, most or all of the amino acids outside the CDR domains have been replaced with amino acids from human immunoglobulins, whereas some, most or all amino acids within one or more CDR regions are unchanged. Small additions, deletions, insertions, substitutions or modifications of amino acids are permissible as long as they do not abrogate the ability of the antibody to bind to a particular antigen. A “humanized” antibody retains an antigenic specificity similar to that of the original antibody.

A “chimeric antibody” refers to an antibody in which the variable regions are derived from one species and the constant regions are derived from another species, such as an antibody in which the variable regions are derived from a mouse antibody and the constant regions are derived from a human antibody. Chimeric antibodies include, for example, non-human antibodies, such as IgG antibodies, in which the Fc region is replaced by a human Fc region.

A “complementarity-determining region”, or “CDR”, refers to amino acid sequences that, together, define the binding affinity and specificity of the variable region of a native immunoglobulin binding site. There are three CDRs in each of the light and heavy chains of an antibody.

A “framework region”, or “FR”, refers to amino acid sequences interposed between CDRs, typically conserved, that act as the scaffold between the CDRs.

A “constant region” refers to the portion of an antibody molecule that is consistent for a class of antibodies and is defined by the type of light and heavy chains. For example, a light chain constant region can be of the kappa or lambda chain type and a heavy chain constant region can be of one of the five chain isotypes: alpha, delta, epsilon, gamma or mu. This constant region, in general, can confer effector functions exhibited by the antibodies. Heavy chains of various subclasses (such as the IgG subclass of heavy chains) are mainly responsible for different effector functions.

As used herein, an “5T4 targeting agent” may be an antibody as defined herein, e.g., full length antibody, minibody, or nanobody, that binds to any available epitope of 5T4 with a high immunoreactivity. The original description of an anti-5T4 antibody sequence was provided by Hole & Stern (Hole, 1988). An antibody for use as an 5T4 targeting agent according to the presently disclosed invention, such as in preclinical studies, may be produced using the sequence provided by Hole & Stern. According to certain aspects, the 5T4 targeting agent is a humanized antibody against 5T4, such as described in U.S. Pat. Nos. 7,074,909 and 8,044,178.

UniProtKB accession no. Q13641 provides an amino acid sequence of human 5T4 (SEQ ID NO:1) which sequence or a portion thereof, such as an extracellular sequence portion, may be used to produce 5T4-specific antibodies, such as monoclonal antibodies, according to methods known in the art. In this sequence, residues 32-355 constitute the extracellular domain, residues 356-376 constitute the transmembrane domain, and residues 377-420 constitute the intracellular domain of the 5T4 protein.

According to certain aspects, the 5T4 targeting agent may, for example, be an antibody selected from: the antibody component of Naptumomab estafenatox; ADC MEDI0641, i.e., “5T4_0108” or any 5T4-specific antibodies disclosed in International Pub. No. WO2015155345A1; ALG.APV-527 or any of the 5T-4 specific scFv domain(s) thereof or 5T4-specific antibodies generally disclosed in U.S. Pat. No. 10,239,949; bispecific Tb535 or the component thereof or any 5T4 binding domains disclosed in U.S. Pat. No. 10,941,207; mAb H6 (the antibody component of H6-DM5); the scFv component (SEQ ID NO:2) of ASN004; ZV05 (the antibody component of ADC ZV0508) disclosed in U.S. Pub. No. 20190374651; any of the anti-5TF monoclonal antibodies disclosed in U.S. Pat. No. 8,759,495; and any of the 5T4-binding antibodies disclosed in U.S. Pub. No. 20110052577. The following ZV05 amino acid sequences are provided: heavy chain variable region (SEQ ID NO:3); light chain variable region (SEQ ID NO:4); heavy chain sequence (SEQ ID NO:5); and light chain sequence (SEQ ID NO:6). The 5T4 targeting agent may, for example, also be anti-extracellular domain rabbit antibody EPR5529 (Catalog #ab201711; Abcam, Waltham, MA, USA), or a chimeric or humanized form thereof. See also Table 4 which discloses additional antibodies and antibody-drug conjugates, wherein the anti-5T4 portions provide 5T4 targeting agents that may be used in the various aspects of the invention.

Naptumomab estafenatox, a recombinant fusion protein of a mouse IgG mAb Fab fragment recognizing human 5T4 and the superantigen staphylococcal enterotoxin A/E-120 fused to the C terminus of the heavy chain, which can be produced using E. coli, is exemplary of chimeric 5T4-targeting antibody-superantigen proteins that can be radiolabeled for use as radiolabeled 5T4-targeting agents in the various aspects of the invention. Naptumomab estafenatox is commercially available from Creative Bioloabs (Catalog #TAB-118 F(E); Shirley, NY, USA). Naptumomab estafenatox is held together by non-covalent interactions between its Fab heavy and light chain components. Its superantigen component was engineered to have low binding to human antibodies and MEW Class II. Naptumomab estafenatox itself can induce T-cell mediated killing of cancer cells at concentrations around 10 μM. The amino acid sequences of the light and heavy chains of Naptumomab estafenatox and of related fusion proteins, for example, having different anti-5T4 antibody components and/or different staphylococcal enterotoxin sequence components, are fully described in U.S. Pat. Nos. 7,615,225, 10,314,910 and U.S. Pub. No. 20200101160, each of which is incorporated by reference in its entirety herein.

SEQ ID NO:7 is a wild-type staphylococcal enterotoxin E (SEE) sequence and SEQ ID NO:8 is a wild-type staphylococcal enterotoxin A (SEA) sequence, each which can be modified for use in a chimeric antibody-enterotoxin 5T4 targeting agent that may be radiolabeled for use as a radiolabeled 5T4 targeting agents in any of the aspects of the invention. Suitable modifications over these wild-type sequences that may be used include for example, K79, K81, K83 and D227 or K79, K81, K83, K84 and D227, or, for example, K79E, K81E, K83S and D227S or K79E, K81E, K83S, K84S and D227A. The modified enterotoxin used may, for example, be SEA/E-120 (SEQ ID NO:9) or SEA_{D227A} (SEQ ID NO:10). SEQ ID NO:16 is another modified enterotoxin sequence that may be used. Exemplary 5T4-binding single chain chimeric antibody-superantigen fusion proteins that may be radiolabeled and used in the various aspects of the invention include C215Fab-SEA (SEQ ID NO:11), 5T4Fab-SEA_{D227A} (SEQ ID NO:12), and 5T4Fab-SEA/E-120 (SEQ ID NO:13). Naptumomab estafenatox is SEQ ID NO:14 (chimeric heavy chain component) non-covalently bound to SEQ ID NO:15 (light chain component). In related detail, the heavy chain includes a 5T4 Fab heavy chain component (corresponding to residues 1 to 222 of SEQ ID NO:13), the SEA/E-120 superantigen (corresponding to residues 226 to 458 of SEQ ID NO:13), and a GGP tripeptide linker (corresponding to residues 223-225 of SEQ ID NO:13) covalently linking the Fab heavy chain and SEA/E-120 components. The light chain includes residues 459 to 672 of SEQ ID NO:13.

5T4 targeting agents that may be radiolabeled for use in any of the various aspects of the invention include but are not limited to C215Fab-SEA (SEQ ID NO:11), 5T4Fab-SEAD227A (SEQ ID NO:12), 5T4Fab-SEA/E-120 (SEQ ID NO:13); SEQ ID NO:14, Naptumomab estafenatox reported as SEQ ID NO:14 (heavy chain component) non-covalently associated with SEQ ID NO:15 (light chain component), an antibody including the Fab component of Naptumomab estafenatox, an antibody including the heavy chain component of any one of SEQ ID NOS:11-14 (for example, lacking the enterotoxin components thereof), a antibody including the heavy chain component of any one of SEQ ID NOS:11-14 and an associated light chain component such as the light chain component of SEQ ID NO:15, a antibody (such as a single- or multi-chain antibody, such as but not limited to a human or humanized IgG, such as IgG1) including a heavy chain component that includes 2 or 3 of the heavy chain CDRs present in any one of SEQ ID NOS:11-14, Naptumomab estafenatox modified by covalent linkage of the heavy chain and light chain components to each other by disulfide bonds (between cysteine residues in the manner of a Fab) and/or by a bifunctional thiol reactive crosslinking agent which may include and introduce a chelating moiety (such as DOTA), any of the chimeric 5T4-binding superantigen fusion proteins of U.S. Pat. Nos. 7,615,225, 10,314,910 and U.S. Pub. No. 20200101160, any of the preceding targeting agents in which the antibody component(s) is/are humanized, any of the preceding in which an amino acid (for example, of a heavy chain and/or of a light chain) is substituted to a cysteine, and any of the preceding further including at least one additional amino acid (for example, in a heavy chain and/or in a light chain thereof) of which at least one is a cysteine and/or at least one is a lysine (for example, one or more additional C-terminal amino acids of which one or more are cysteines and/or of which one or more are lysines).

Light chain variations that may also be used include but are not limited to SEQ ID NO:17 (SEQ ID NO:15 with C-terminal lysine), SEQ ID NO:18 (SEQ ID NO: 15 with C-terminal cysteine), SEQ ID NO:19 (SEQ ID NO:15 with C-terminal lys-cys), and a light chain including 2 or 3 of the CDRs of the light chain of SEQ ID NO:15.

Further 5T4 targeting agents that may be radiolabeled for use in any of the various aspects of the invention include but are not limited to the following and 5T4 targeting proteins that include the following:
SEQ ID NO:20 [heavy chain portion only of SEQ ID NO:14];
SEQ ID NO:21 [SEQ ID NO:14 including linker, excluding enterotoxin portion];
SEQ ID NO:22 [SEQ ID NO:20 with C-terminal lysine];
SEQ ID NO:23 [SEQ ID NO:21 with C-terminal lysine];
SEQ ID NO:24 [SEQ ID NO:20 with C-terminal cysteine];
SEQ ID NO:25 [SEQ ID NO:21 with C-terminal cysteine];
SEQ ID NO:26 [SEQ ID NO:20 with C-terminal lys-cys];
SEQ ID NO:27 [SEQ ID NO:21 with C-terminal lys-cys];
SEQ ID NO:28 [5T4Fab-SEA/E-120 (SEQ ID NO:13) with C-terminal lysine];
SEQ ID NO:29 [SEQ ID NO:14 with C-terminal lysine];
SEQ ID NO:30 [5T4Fab-SEA/E-120 (SEQ ID NO:13) with C-terminal cysteine];
SEQ ID NO:31 [SEQ ID NO:14 with C-terminal cysteine];
SEQ ID NO:32 [5T4Fab-SEA/E-120 (SEQ ID NO:13) with C-terminal lys-cys];
SEQ ID NO:33 [SEQ ID NO:14 with C-terminal lys-cys]; and
any of SEQ ID NOS:14, 20-27, 29, 31 and 33 in non-covalent association with any of light chain SEQ ID NOS:15 and 17-19 to form a Fab without covalent cross-linking between chains, or a disulfide-bonded (cross-linked) form of such a Fab, or a form of such a Fab covalently cross-linked by a bifunctional cross-linker that may include a chelator such as DOTA.

Where the 5T4 targeting agent includes a heavy chain and a light chain, one or both of the heavy chain and the light chain may be radiolabeled, for example, by chemical conjugation to a chelator, such as DOTA, and chelation of a radionuclide, such as ²²⁵Ac or ¹⁷⁷Lu, by the chelator.

According to certain aspects, the 5T4 targeting agent may be a peptide or small molecule that binds to 5T4.

As used herein, “Immunoreactivity” refers to a measure of the ability of an immunoglobulin to recognize and bind to a specific antigen. “Specific binding” or “specifically binds” or “binds” refers to an antibody binding to an antigen or an epitope within the antigen with greater affinity than for other antigens. Typically, the antibody binds to the antigen or the epitope within the antigen with an equilibrium dissociation constant (K_{D}) of about 1×10⁻⁸ M or less, for example about 1×10⁻⁹ M or less, about 1×10⁻¹⁰ M or less, about 1×10⁻¹¹ M or less, or about 1×10⁻¹² M or less, typically with the K_{D} that is at least one hundred fold less than its K_{D} for binding to a nonspecific antigen (e.g., BSA, casein). The dissociation constant may be measured using standard procedures. Antibodies that specifically bind to the antigen or the epitope within the antigen may, however, have cross-reactivity to other related antigens, for example to the same antigen from other species (homologs), such as human or monkey, for example Macaca fascicularis (cynomolgus, cyno), Pan troglodytes (chimpanzee, chimp) or Callithrix jacchus (common marmoset, marmoset).

An “epitope” refers to the target molecule site (e.g., at least a portion of an antigen) that is capable of being recognized by, and bound by, a targeting agent such as an antibody, antibody fragment, Fab fragment, or aptamer. For a protein antigen, for example, this may refer to the region of the protein (i.e., amino acids, and particularly their side chains) that is bound by the antibody. Overlapping epitopes include at least one to five common amino acid residues. Methods of identifying epitopes of antibodies are known to those skilled in the art and include, for example, those described in Antibodies, A Laboratory Manual, Cold Spring Harbor Laboratory, Ed Harlow and David Lane (1988).

As used herein, the terms “proliferative disorder” and “cancer” may be used interchangeably and may include, without limitation, a solid cancer (e.g., a tumor). “Solid cancers” include, without limitation, bone cancer, pancreatic cancer, skin cancer, cancer of the head or neck, cutaneous or intraocular malignant melanoma, uterine cancer, ovarian cancer, prostate cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, pediatric tumors, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, environmentally-induced cancers including those induced by asbestos.

According to certain aspects, the solid cancer may be breast cancer, gastric cancer, bladder cancer, cervical cancer, endometrial cancer, skin cancer, stomach cancer, testicular cancer, esophageal cancer, bronchioloalveolar cancer, prostate cancer, colorectal cancer, ovarian cancer, cervical epidermoid cancer, pancreatic cancer, lung cancer such as non-small cell lung carcinoma, renal cancer, head and neck cancer such as head and neck squamous cell cancer, or any combination thereof.

According to certain aspects, the 5T4 targeting agent may be labeled with a radioisotope. As used herein, a “radioisotope” can be an alpha-emitting isotope, a beta-emitting isotope, and/or a gamma-emitting isotope. Examples of radioisotopes include the following: ¹³¹I, ¹²⁵I, ¹²³I, ⁹⁰Y, ¹⁷⁷Lu, ¹⁸⁶Re, ¹⁸⁸Re, ⁸⁹Sr, ¹⁵³Sm, ³²P, ²²⁵Ac, ²¹³Bi, ²¹³Po, ²¹¹At, ²¹²Bi, ²¹³Bi, ²²³Ra, ²²⁷Th, ¹⁴⁹Tb, ¹³⁷Cs, ²¹²Pb and ¹⁰³Pd. Methods for affixing a protein such as an antibody or antibody fragment (i.e., “labeling” an antibody with a radioisotope) are well known. Specific methods for labeling are described, for example, in any of U.S. Pat. No. 10,420,851, International Pub. No. WO 2017/155937 and U.S. Provisional Patent Application No. 63/042,651 filed Dec. 9, 2019 and titled “Compositions and methods for preparation of site-specific radioconjugates,” both of which are incorporated by reference herein.

According to certain aspects, the 5T4 targeting agent may be an antibody, peptide, or small molecule radiolabeled with ²²⁵Ac (“²²⁵Ac-labeled”), and the effective amount may, for example, be below 50.0 μCi/kg (i.e., where the amount of ²²⁵Ac administered to the subject delivers a radiation dose of below 50.0 μCi per kilogram of subject's body weight). According to certain aspects, when the 5T4 targeting agent is ²²⁵Ac-labeled, the effective amount is below 50 μCi/kg, 40 μCi/kg, 30 μCi/kg, 20 μCi/kg, 10 μCi/kg, 5 μCi/kg, 4 μCi/kg, 3 μCi/kg, 2 μCi/kg, 1 μCi/kg, or even 0.5 μCi/kg. According to certain aspects, when the 5T4 targeting agent is ²²⁵Ac-labeled, the effective amount is at least 0.05 μCi/kg, or 0.1 μCi/kg, 0.2 μCi/kg, 0.3 μCi/kg, 0.4 μCi/kg, 0.5 μCi/kg, 1 μCi/kg, 2 μCi/kg, 3 μCi/kg, 4 μCi/kg, 5 μCi/kg, 6 μCi/kg, 7 μCi/kg, 8 μCi/kg, 9 μCi/kg, 10 μCi/kg, 12 μCi/kg, 14 μCi/kg, 15 μCi/kg, 16 μCi/kg, 18 μCi/kg, 20 μCi/kg, μCi/kg, or 40 μCi/kg. According to certain aspects, the ²²⁵Ac-labeled 5T4 targeting agent may be administered at a dose that includes any combination of upper and lower limits as described herein, such as from at least 0.1 μCi/kg to below 5 μCi/kg, or from at least 5 μCi/kg to below 20 μCi/kg.

According to certain aspects, the 5T4 targeting agent may be an antibody, peptide, or small molecule that is ²²⁵Ac-labeled, and the effective amount may be below 2 mCi (i.e., wherein the ²²⁵Ac is administered to the subject in a non-weight-based dosage). According to certain aspects, the effective dose of the ²²⁵Ac-labeled 5T4 targeting agent may be below 1 mCi, such as 0.9 mCi, 0.8 mCi, 0.7 mCi, 0.6 mCi, 0.5 mCi, 0.4 mCi, 0.3 mCi, 0.2 mCi, 0.1 mCi, 90 μCi, 80 μCi, 70 μCi, 60 μCi, 50 μCi, 40 μCi, 30 μCi 20 μCi, 10 μCi, or 5 μCi. The effective amount of ²²⁵Ac-labeled 5T4 targeting agent may be at least 2 μCi, such as at least 5 μCi, 10 μCi, μCi, 30 μCi, 40 μCi, 50 μCi, 60 μCi, 70 μCi, 80 μCi, 90 μCi, 100 μCi, 200 μCi, 300 μCi, 400 μCi, 500 μCi, 600 μCi, 700 μCi, 800 μCi, 900 μCi, 1 mCi, 1.1 mCi, 1.2 mCi, 1.3 mCi, 1.4 mCi, or 1.5 mCi. According to certain aspects, the ²²⁵Ac-labeled 5T4 targeting agent may be administered at a dose that includes any combination of upper and lower limits as described herein, such as from at least 2 μCi to below 1 mCi, or from at least 2 μCi to below 250 μCi, or from 75 μCi to below 400 μCi.

According to certain aspects, the ²²⁵Ac-labeled 5T4 targeting agent includes a single dose that delivers less than 12 Gy, or less than 8 Gy, or less than 6 Gy, or less than 4 Gy, or less than 2 Gy, such as doses of 2 Gy to 8 Gy, to the subject, such as predominantly to the targeted solid tumor.

According to certain aspects, the 5T4 targeting agent may be an antibody, peptide, or small molecule radiolabeled with ¹⁷⁷Lu (“¹⁷⁷Lu-labeled”), and the effective amount may, for example, be below 1 mCi/kg (i.e., where the amount of ¹⁷⁷Lu-labeled antibody administered to the subject delivers a radiation dose of below 1000 mCi per kilogram of subject's body weight). According to certain aspects, when the antibody is ¹⁷⁷Lu-labeled, the effective amount is below 900 μCi/kg, 800 μCi/kg, 700 μCi/kg, 600 μCi/kg, 500 μCi/kg, 400 μCi/kg, 300 μCi/kg, 200 μCi/kg, 150 μCi/kg, 100 μCi/kg, 80 μCi/kg, 60 μCi/kg, 50 μCi/kg, 40 μCi/kg, 30 μCi/kg, 20 μCi/kg, 10 μCi/kg, 5 μCi/kg, or 1 μCi/kg. According to certain aspects, the effective amount of the ¹⁷⁷Lu-labeled antibody is at least 1 μCi/kg, 2.5 μCi/kg, 5 μCi/kg, 10 μCi/kg, 20 μCi/kg, 30 μCi/kg, 40 μCi/kg, 50 μCi/kg, 60 μCi/kg, 70 μCi/kg, 80 μCi/kg, 90 μCi/kg, 100 μCi/kg, 150 μCi/kg, 200 μCi/kg, 250 μCi/kg, 300 μCi/kg, 350 μCi/kg, 400 μCi/kg or 450 μCi/kg. According to certain aspects, an ¹⁷⁷Lu-labeled antibody may be administered at a dose that includes any combination of upper and lower limits as described herein, such as from at least 5 mCi/kg to below 50 μCi/kg, or from at least 50 mCi/kg to below 500 μCi/kg.

According to certain aspects, the 5T4 targeting agent may be an antibody that is ¹⁷⁷Lu-labeled, and the effective amount may, for example, be below 45 mCi, such as below 40 mCi, 30 mCi, 20 mCi, 10 mCi, 5 mCi, 3.0 mCi, 2.0 mCi, 1.0 mCi, 800 μCi, 600 μCi, 400 μCi, 200 μCi, 100 μCi, or 50 Xi. The effective amount of ¹⁷⁷Lu-labeled 5T4 targeting agent may be at least 10 μCi, such as at least 25 μCi, 50 μCi, 100 μCi, 200 μCi, 300 μCi, 400 μCi, 500 μCi, 600 μCi, 700 μCi, 800 μCi, 900 μCi, 1 mCi, 2 mCi, 3 mCi, 4 mCi, 5 mCi, 10 mCi, 15 mCi, 20 mCi, 25 mCi, 30 mCi. According to certain aspects, an ¹⁷⁷Lu-labeled antibody may be administered at a dose that includes any combination of upper and lower limits as described herein, such as from at least 10 mCi to below 30 mCi, or from at least 100 μCi to below 3 mCi, or from 3 mCi to below 30 mCi.

According to certain aspects, the 5T4 targeting agent may be an antibody, peptide, or small molecule radiolabeled with ¹³¹I (“¹³¹I-labeled”), and the effective amount may, for example, be below, for example, 1200 mCi (i.e., where the amount of ¹³¹I administered to the subject delivers a total body radiation dose of below 1200 mCi in a non-weight-based dose). According to certain aspects, the effective amount of the ¹³¹I-labeled targeting agent may be below 1100 mCi, below 1000 mCi, below 900 mCi, below 800 mCi, below 700 mCi, below 600 mCi, below 500 mCi, below 400 mCi, below 300 mCi, below 200 mCi, below 150 mCi, or below 100 mCi. According to certain aspects, the effective amount of the ¹³¹I-labeled targeting agent may be below 200 mCi, such as below 190 mCi, 180 mCi, 170 mCi, 160 mCi, 150 mCi, 140 mCi, 130 mCi, 120 mCi, 110 mCi, 100 mCi, 90 mCi, 80 mCi, 70 mCi, 60 mCi, or 50 mCi. According to certain aspects, the effective amount of the ¹³¹I-labeled targeting agent may be at least 1 mCi, such as at least 2 mCi, 3 mCi, 4 mCi, 5 mCi, 6 mCi, 7 mCi, 8 mCi, 9 mCi, 10 mCi, mCi, 30 mCi, 40 mCi, 50 mCi, 60 mCi, 70 mCi, 80 mCi, 90 mCi, 100 mCi, 110 mCi, 120 mCi, 130 mCi, 140 mCi, 150 mCi, 160 mCi, 170 mCi, 180 mCi, 190 mCi, 200 mCi, 250 mCi, 300 mCi, 350 mCi, 400 mCi, 450 mCi, 500 mCi. According to certain aspects, an ¹³¹I-labeled targeting agent may be administered at a dose that includes any combination of upper and lower limits as described herein, such as from at least 1 mCi to below 100 mCi, or at least 10 mCi to below 200 mCi.

While select radionuclides have been disclosed in detail herein, any from the list provided above are contemplated for labeling the 5T4 targeting agents that are part of the presently disclosed invention.

As used herein, a composition including a 5T4 targeting agent includes a “patient specific composition” that includes both a radionuclide labeled portion and a non-labeled portion. According to certain aspects of the present invention, when the 5T4 targeting agent is labeled with a radioisotope, the majority of the targeting agent (antibody, antibody fragment, etc.) administered to a patient typically consists of non-labeled targeting agent, with the minority being the labeled targeting agent. The ratio of labeled to non-labeled targeting agent can be adjusted using known methods. According to certain aspects of the present invention, the patient specific composition may include the 5T4 targeting agent in a ration of labeled:unlabeled 5T4 targeting agent of from about 0.01:10 to 1:1, such as 0.1:10 to 1:1 labeled:unlabeled.

Accordingly to certain aspects of the present invention, the 5T4 targeting agent may be provided in a total protein amount of up to 100 mg, such as up to 60 mg, such as 5 mg to 45 mg, or a total protein amount of from 0.001 mg/kg patient weight to 3.0 mg/kg patient weight, such as from 0.005 mg/kg patient weight to 2.0 mg/kg patient weight, or from 0.01 mg/kg patient weight to 1 mg/kg patient weight, or from 0.1 mg/kg patient weight to 0.6 mg/kg patient weight, or 0.3 mg/kg patient weight, or 0.4 mg/kg patient weight, or 0.5 mg/kg patient weight, or 0.6 mg/kg patient weight.

This inventive combination of a labeled fraction and a non-labeled fraction of the antibody or other biologic delivery vehicle allows the composition to be tailored to a specific patient, wherein each of the radiation dose and the protein dose of the antibody or other biologic delivery vehicle are personalized to that patient based on at least one patient specific parameter. As such, each vial of the composition may be made for a specific patient, where the entire content of the vial is delivered to that patient in a single dose. When a treatment regime calls for multiple doses, each dose may be formulated as a patient specific dose in a vial to be administered to the patient as a “single dose” (i.e., full contents of the vial administered at one time). The subsequent dose may be formulated in a similar manner, such that each dose in the regime provides a patient specific dose in a single dose container. One of the advantages of the disclosed composition is that there will be no left-over radiation that would need to be discarded or handled by the medical personnel, e.g., no dilution, or other manipulation to obtain a dose for the patient. When provided in a single dose container, the container is simply placed in-line in an infusion tubing set for infusion to the patient. Moreover, the volume can be standardized so that there is a greatly reduced possibility of medical error (i.e., delivery of an incorrect dose, as the entire volume of the composition is to be administered in one infusion).

Thus, according to certain aspects, the 5T4 targeting agent may be provided as a single dose composition tailored to a specific patient, wherein the amount of labeled and unlabeled 5T4 targeting agent in the composition may depend on and/or be determined based on at least a patient weight, age, and/or disease state or health status, for example, as described in U.S. Pat. No. 10,736,975 and International Pub. No. WO 2016/187514.

As used herein, the terms “subject” and “patient” are interchangeable and include, without limitation, a mammal such as a human, a non-human primate, a dog, a cat, a horse, a sheep, a goat, a cow, a rabbit, a pig, a rat and a mouse. Where the subject is human, the subject can be of any age. For example, the subject can be 60 years or older, 65 or older, 70 or older, 75 or older, 80 or older, 85 or older, or 90 or older. Alternatively, the subject can be 50 years or younger, 45 or younger, 40 or younger, 35 or younger, 30 or younger, 25 or younger, or 20 or younger. For a human subject afflicted with cancer, the subject can be newly diagnosed, or relapsed and/or refractory, or in remission.

As used herein, “treating” a subject afflicted with a cancer shall include, without limitation, (i) slowing, stopping or reversing the cancer's progression, (ii) slowing, stopping or reversing the progression of the cancer's symptoms, (iii) reducing the likelihood of the cancer's recurrence, and/or (iv) reducing the likelihood that the cancer's symptoms will recur. According to certain preferred aspects, treating a subject afflicted with a cancer means (i) reversing the cancer's progression, ideally to the point of eliminating the cancer, and/or (ii) reversing the progression of the cancer's symptoms, ideally to the point of eliminating the symptoms, and/or (iii) reducing or eliminating the likelihood of relapse (i.e., consolidation, which ideally results in the destruction of any remaining cancer cells).

“Chemotherapeutic”, in the context of this invention, shall mean a chemical compound which inhibits or kills growing cells and which can be used or is approved for use in the treatment of cancer. Exemplary chemotherapeutic agents that may be used include cytostatic agents which prevent, disturb, disrupt or delay cell division at the level of nuclear division or cell plasma division. Such agents may stabilize microtubules, such as taxanes, in particular docetaxel or paclitaxel, and epothilones, in particular epothilone A, B, C, D, E, and F, or may destabilize microtubules such as vinca alkaloids, in particular vinblastine, vincristine, vindesine, vinflunine, and vinorelbine.

As used herein, “5T4” refers to the 5T4 oncofetal antigen, a 72 kDa highly glycosylated trans-membrane glycoprotein including a 42 kDa non-glycosylated core (see U.S. Pat. No. 5,869,053) that is also referred to as Trophoblast Glycoprotein or TPBG, and Wnt-Activated Inhibitory Factor 1 or WAIF1. The 5T4 antigen is a human protein encoded by the TPBG gene and is an antagonist of Wnt/β-catenin signaling pathway. Human 5T4 is expressed in numerous cancer types, including but not limited to carcinomas of the bladder, breast, cervix, colon, endometrium, kidney, lung, esophagus, ovary, prostate, pancreas, skin, stomach, head and neck, and testes. Highly tumorigenic cells, also called cancer stem cells or tumor-initiating cells have been shown to have high levels of 5T4 expression (WO2010/111659). 5T4 is substantially absent from normal adult human tissues.

“Therapeutically effective amount” or “effective amount” refers to an amount effective, at dosages and for periods of time necessary, to achieve a therapeutic result. A therapeutically effective amount may vary according to factors such as the disease state, age, sex, and weight of the individual, and the ability of a therapeutic or a combination of therapeutics to elicit a desired response in the individual. Exemplary indicators of an effective therapeutic or combination of therapeutics include, for example, improved well-being of the patient, reduction in a tumor burden, arrested or slowed growth of a tumor, and/or absence of metastasis of cancer cells to other locations in the body. According to certain aspects, “therapeutically effective amount” or “effective amount” refers to an amount of the 5T4 targeting agent that may deplete or cause a reduction in the overall number of cells expressing 5T4, or an amount of the 5T4 targeting agent that may inhibit growth of cells expressing 5T4.

As used herein, “depleting”, with respect to cells expressing 5T4, shall mean to lower the population of at least one type of cells that express or overexpress 5T4 (e.g., 5T4-positive cells in a solid tumor or circulating in a subject's blood). According to certain aspects of this invention, a decrease is determined by comparison of the numbers of 5T4-positive cells in the subject's blood or in a tissue biopsy, such as from the solid tumor, before and after initiation of treatment with the 5T4 targeting agent. As such, and by way of example, a subject's 5T4-positive cells may be considered to be depleted if the population is lowered, such as by at least 10%, 20%, 30%, 40%, 50%, 60%, 70%, 80%, 90% or 99%.

“Inhibits growth” refers to a measurable decrease or delay in the growth of a malignant cell or tissue (e.g., tumor) in vitro or in vivo when contacted with a therapeutic or a combination of therapeutics or drugs, when compared to the decrease or delay in the growth of the same cells or tissue in the absence of the therapeutic or the combination of therapeutic drugs. Inhibition of growth of a malignant cell or tissue in vitro or in vivo may be at least about 10%, 20%, 30%, 40%, 50%, 60%, 70%, 80%, 90%, 95%, 96%, 97%, 98%, 99%, or 100%.

The term “immune checkpoint therapy” refers to a molecule capable of modulating the function of an immune checkpoint protein in a positive or negative way (in particular, the interaction between an antigen presenting cell (APC) such as a cancer cell and an immune T effector cell). The term “immune checkpoint” refers to a protein directly or indirectly involved in an immune pathway that under normal physiological conditions is crucial for preventing uncontrolled immune reactions and thus for the maintenance of self-tolerance and/or tissue protection. The one or more immune checkpoint therapies described herein may independently act at any step of the T cell-mediated immunity including clonal selection of antigen-specific cells, T cell activation, proliferation, trafficking to sites of antigen and inflammation, execution of direct effector function and signaling through cytokines and membrane ligands. Each of these steps is regulated by counterbalancing stimulatory and inhibitory signals that fine tune the response.

In the context of the present invention, the term immune checkpoint therapy encompasses therapies such as antibodies capable of down-regulating at least partially the function of an inhibitory immune checkpoint (antagonist) and/or up-regulating at least partially the function of a stimulatory immune checkpoint (agonist). As example, an immune checkpoint therapy may refer to an antibody against an immune checkpoint inhibitor (ICI) that may be upregulated in certain cancers, and thus may inhibit the function of the ICI.

The term “DDRi” refers to an inhibitor of a DNA damage response pathway protein, of which a PARPi is an example. The term “PARPi” refers to an inhibitor of poly(ADP-ribose) polymerase. In the context of the present invention, the term PARPi encompasses molecules that may bind to and inhibitor the function of poly(ADP-ribose) polymerase, such as antibodies.

As used herein, administering to a subject either or both of an immune checkpoint therapy and DDRi “in conjunction with” a 5T4 targeting agent means administering the immune checkpoint therapy and/or DDRi before, during and/or after administration of the 5T4 targeting agent. This administration includes, without limitation, the following scenarios: (i) the immune checkpoint therapy and/or DDRi is administered first, and the 5T4 targeting agent is administered second; (ii) the immune checkpoint therapy and/or DDRi is administered concurrently with the 5T4 targeting agent (e.g., the DDRi is administered orally once per day for n days, and the 5T4 targeting agent is administered intravenously in a single dose on one of days 2 through n−1 of the DDRi regimen); (iii) the immune checkpoint therapy and/or DDRi is administered concurrently with the 5T4 targeting agent (e.g., the DDRi is administered orally for a duration of greater than one month, such as orally once per day for 35 days, 42 days, 49 days, or a longer period during which the cancer being treated does not progress and during which the DDRi does not cause unacceptable toxicity, and the 5T4 targeting agent is administered intravenously in a single dose on a day within the first month of the DDRi regimen); and (iv) the 5T4 targeting agent is administered first (e.g., intravenously in a single dose or a plurality of doses over a period of weeks), and the immune checkpoint therapy and/or DDRi is administered second (e.g., the DDRi is administered orally once per day for 21 days, 28 days, 35 days, 42 days, 49 days, or a longer period during which the cancer being treated does not progress and during which the DDRi does not cause unacceptable toxicity). Additional permutations that would be obvious to one of skill in the art are possible and within the scope of the presently claimed invention.

“Synergistic combinations” include combinations of agents and/or therapies that provide a therapeutic effect that is comparable to the effectiveness of a monotherapy, while reducing adverse side effects, e.g. damage to non-targeted tissues, immune status, and other clinical indicia, and combinations that provide greater than additive effectiveness versus that seen with the individual agents and/or therapies when used in corresponding amounts/strengths, in one or more relevant parameters such as but not limited to reduction in total tumor/cancer cell number, reduction in tumor size, increase in the length of time to relapse, and other indicia of patient health or treatment success.

An “article of manufacture” indicates a package containing materials useful for the treatment, prevention and/or diagnosis of the disorders described herein. The article of manufacture may include a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is a 5T4 targeting agent according to aspects of the presently disclosed invention.

A “label” or “package insert” is used to refer to instructions customarily included in commercial packages of therapeutic products that contain information about the indications, usage, dosage, administration, combination therapy, contraindications and/or warnings concerning the use of such therapeutic products. As used herein, a label may indicate that the composition is used for treating a 5T4-positive cancer and may optionally indicate administration routes and/or methods. Moreover, the article of manufacture may include (a) a first container with a composition contained therein, wherein the composition includes 5T4 targeting agent; and (b) a second container with a composition contained therein, wherein the composition includes a further cytotoxic or otherwise therapeutic agent according to aspects of the presently disclosed invention. Alternatively, or additionally, the article of manufacture may further include a second (or third) container including a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Throughout this application, various patents, patent applications and other publications are cited. The disclosure of each of these publications is hereby incorporated by reference herein.

Unless otherwise defined, all technical and scientific terms used herein have the same meaning as commonly understood by one of ordinary skill in the art to which the present invention belongs. Although methods and materials similar or equivalent to those described herein can be used in the practice or testing described herein, suitable methods and materials are described below.

### ASPECTS OF THE INVENTION

According to certain aspects of the presently disclosed invention, therapeutic methods for treating 5T4-positive cancers are provided. The methods may include diagnostic steps to identify if the patient has a 5T4 positive cancer, such as by identifying 5T4 positive cells within solid tumors or circulating in a blood sample from the patient.

According to certain aspects, the therapeutic methods include administration of a radiolabeled 5T4 targeting agent, such as a radiolabeled antibody, peptide, or small molecule that targets 5T4, either alone or in combination with an additional method of treatment. According to certain aspects, the additional method of treatment may be any one or more of administration of an immune checkpoint therapy, a DDRi, a chemotherapeutic agent, or radiation therapy.

According to certain aspect, the 5T4 targeting agent may be administered to the patient in a patient specific composition in one or more doses.

According to certain aspects, the patient may be monitored at intervals during the therapy for the presence of 5T4 positive cells to evaluate the reduction in 5T4-positive cells. Detecting a decreased number of the 5T4-positive cells after treatment with the 5T4 targeting agent, as compared to the number of 5T4-positive cells prior to treatment may indicate effectiveness of the 5T4 targeting agent in treating a 5T4-positive cancer in the mammalian subject.

According to certain aspects, the method of treating cancer includes identifying a patient that has a 5T4-positive cancer by identifying 5T4-positive cells and administering to the patient an effective amount of a 5T4 targeting agent, either alone or in combination with an additional method of treatment. According to certain aspects, the additional method of treatment may be any one or more of administration of an immune checkpoint therapy, a DDRi, a chemotherapeutic agent, or radiation therapy.

According to certain aspects, the chemotherapeutic agent is one that has not been found to be refractory for treatment of the cancer. According to other aspects, the chemotherapeutic agent is one that has been found to be refractory for treatment of the cancer.

According to certain aspects, the 5T4 targeting agent can be administered to a patient that has also undergone a treatment, such as surgery for treatment of the cancer, such as to remove all or a portion of a solid tumor.

5T4 Targeting Agent Toxicity

Prior work in the field has demonstrated little to no success using an unlabeled 5T4-targeting antibody (Boghaert, 2008). Thus, the standard methods by which antibody therapeutics may affect a response, such as antibody-dependent cellular cytotoxicity (ADCC), complement-dependent cytotoxicity (CDC), and apoptosis, are not sufficient for 5T4-targeting agents. Further, arming the 5T4 antibodies with toxins, i.e., antibody drug-conjugates (ADC), have also provided limited success (Boghaert, 2008; Borghaei 2009; Cheng 2004; Harper, 2017; Kerk, 2017; Sapra, 2018; Shaw 2007; Shi, 2019; Wang, 2018).

An object of the presently disclosed invention is to provide a 5T4 targeting agent that may be useful in diagnostic assays and effective in therapeutic interventions, such as for the treatment of 5T4-positive cancers. Proposed methods by which the presently disclosed radiolabeled 5T4 targeting agents affect a therapeutic response includes targeted DNA damage from the ionizing radiation provided by the radiolabel (e.g., double strand DNA breaks).

Thus, the present invention contemplates the treatment of proliferative diseases or disorders, such as solid tumors, with a radiolabeled 5T4 targeting agent that functions to target ionizing radiation to cells expressing 5T4, and thus induce DNA damage in those cells.

The present invention further contemplates methods of treating a proliferative disease or disorder which includes administration of a multi-specific antibody against two or more epitopes of 5T4, or against an epitope of 5T4 and an epitope of one or more additional different antigens.

The additional different antigens may be antigens differentially expressed on cells involved in various diseases or disorders, and/or cells involved in solid tumors. For example, the additional different antigens may be selected from the group including mesothelin, TSHR, CD19, CD123, CD22, CD30, CD33, CD45, CD171, CD138, CS-1, CLL-1, GD2, GD3, B-cell maturation antigen (BCMA), T-Ag, Tn-Ag, prostate specific membrane antigen (PSMA), ROR1, FLT3, FAP, TAG72, CD38, CD44v6, CEA, EPCAM, B7H3, KIT, IL-13Ra2, interleukin-11 receptor a (IL-11Ra), PSCA, PRSS21, VEGFR2, LewisY, CD24, platelet-derived growth factor receptor-beta (PDGFR-beta), SSEA-4, CD20, Folate receptor alpha (FRa), ERBB2 (HER2/neu), ERBB3 (HER3), MUC1, epidermal growth factor receptor (EGFR), EGFRvIII, NCAM, Prostase, PAP, ELF2M, Ephrin B2, IGF-I receptor, CAIX, LMP2, gp100, bcr-abl, tyrosinase, EphA2, Fucosyl GM1, sLe, GM3, TGS5, HMWMAA, o-acetyl-GD2, Folate receptor beta, TEM1/CD248, TEM7R, CLDN6, GPRC5D, CXORF61, CD97, CD 179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, legumain, HPV E6,E7, MAGE A1, MAGEA3, MAGEA3/A6, ETV6-AML, sperm protein 17, XAGE1, Tie 2, MAD-CT-1, MAD-CT-2, Fos-related antigen 1, prostein, survivin and telomerase, PCTA-1/Galectin 8, KRAS, MelanA/MART1, Ras mutant, hTERT, sarcoma translocation breakpoints, ML-IAP, ERG (TMPRSS2 ETS fusion gene), NA17, PAX3, Androgen receptor, Cyclin B 1, MYCN, RhoC, TRP-2, CYP1B 1, BORIS, SART3, PAX5, OY-TES 1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, GPA7, and IGLL1.

Alternatively, the present invention contemplates methods of treating a proliferative disease or disorder which includes administration of a first antibody against at least one epitope of 5T4, and administration of a second antibody, wherein the second antibody is against a different epitope of 5T4 than the first antibody, or is against an epitope of a different antigen, such as an antigen selected from the list presented above, or an immune checkpoint antigen (see discussion regarding immune checkpoint therapies below), or a DNA damage response antigen.

Such combinations, presented as a multi-specific antibody or more than one monoclonal antibody as indicated above, may deliver a synergistic combination that provides a therapeutic effect which is comparable to the effectiveness of a monotherapy with only an antibody against 5T4, while reducing adverse side effects of the monotherapy. Moreover, the combination may deliver an improved effectiveness over the monotherapy, which may be measured by reduction in the total tumor cell number, increase in the length of time to relapse, and other indicia of patient health.

When the methods include administration of a multi-specific antibody, the first target recognition component may include one of: a first full length heavy chain and a first full length light chain, a first Fab fragment, or a first single-chain variable fragment (scFvs). The second target recognition component may include one of: a second full length heavy chain and a second full length light chain, a second Fab fragment, or a second single-chain variable fragment (scFvs). Moreover, the second target recognition component may be derived from a different epitope of the 5T4 antigen or may be derived from any of the antigens listed above.

The 5T4 targeting agent includes a radioisotope, and any additional antibodies against other antigens may optionally include a radioisotope. According to certain aspects of the present invention, when the immunotherapy includes a bispecific antibody, either one or both of the first target recognition component and the second target recognition component may include a radioisotope.

According to certain aspects of the present invention, the radiolabeled targeting agent may exhibit essentially the same immunoreactivity to the antigen as a control targeting agent, wherein the control targeting agent includes an un-labeled targeting agent against the same epitope of the antigen (i.e., 5T4) as the radiolabeled targeting agent.

According to certain aspects of the present invention, the targeting agent may be labeled with ²²⁵Ac, and may be at least 5-fold more effective at causing cell death of 5T4-positive cells than a control monoclonal antibody, wherein the control monoclonal antibody includes an un-labeled antibody against the same epitope of the antigen as the ²²⁵Ac labeled antibody. For example, a ²²⁵Ac labeled monoclonal antibody may be at least 10-fold more effective, at least 20-fold more effective, at least 50-fold more effective, or at least 100-fold more effective at causing cell death of 5T4-positive cells than the control monoclonal antibody.

According to certain aspects of the present invention, the methods may include administration of labeled and un-labeled (e.g., “naked”) fractions of the 5T4 targeting agent, such as an antibody, antibody fragment, etc. For example, the un-labeled fraction may include the same antibody against the same epitope as the labeled fraction. In this way, the total radioactivity of the antibody may be varied or may be held constant while the overall antibody protein concentration may be held constant or may be varied, respectively. For example, the total protein concentration of un-labeled antibody fraction administered may vary depending on the exact nature of the disease to be treated, age and weight of the patient, identity of the monoclonal antibody, and the label (e.g., radionuclide) selected for labeling of the monoclonal antibody.

According to certain aspects of the present invention, the effective amount of the anti-5T4 antibody is a maximum tolerated dose (MTD) of the anti-5T4 antibody.

According to certain aspects of the methods of the present invention, when more than one antibody is administered, the antibodies may be administered at the same time. As such, according to certain aspects of the present invention, the antibodies may be provided in a single composition. Alternatively, the two antibodies may be administered sequentially. As such, the 5T4 targeting agent may be administered before the second antibody, after the second antibody, or both before and after the second antibody. Moreover, the second antibody may be administered before the 5T4 targeting agent, after the 5T4 targeting agent, or both before and after the 5T4 targeting agent.

According to certain aspects of the methods of the present invention, the 5T4 targeting agent may be administered according to a dosing schedule selected from the group consisting of one every 7, 10, 12, 14, 20, 24, 28, 35, and 42 days throughout a treatment period, wherein the treatment period includes at least two doses.

According to certain aspects of the present invention, the 5T4 targeting agent may be administered according to a dose schedule that includes 2 doses, such as on days 1 and 5, 6, 7, 8, 9, or 10 of a treatment period, or days 1 and 8 of a treatment period.

Administration of the 5T4 targeting agents of the present invention, in addition to other therapeutic agents, may be provided in a number of ways depending upon whether local or systemic treatment is desired and upon the area to be treated. Administration may be intratracheal, intranasal, epidermal and transdermal, oral or parenteral. Parenteral administration includes intravenous, intra-arterial, subcutaneous, intraperitoneal or intramuscular injection or infusion; or intracranial, e.g., intrathecal or intraventricular, administration. In some embodiments a slow-release preparation including the targeting agents(s) and/or other therapeutic agents may be administered. The various agents may be administered as a single treatment or in a series of treatments that continue as needed and for a duration of time that causes one or more symptoms of the cancer to be reduced or ameliorated, or that achieves another desired effect.

The dose(s) may vary, for example, depending upon the identity, size, and condition of the subject, further depending upon the route by which the composition is to be administered and the desired effect. Appropriate doses of a therapeutic agent depend upon the potency with respect to the expression or activity to be modulated. The therapeutic agents can be administered to an animal (e.g., a human) at a relatively low dose at first, with the dose subsequently increased until an appropriate response is obtained.

The radiolabeled 5T4 targeting agent may be administered simultaneously or sequentially with the one or more additional therapeutic agents. Moreover, when more than one additional therapeutic agent is included, the additional therapeutic agents may be administered simultaneously or sequentially with each other and/or with the radiolabeled 5T4 targeting agent.

Radiolabeling the 5T4 Targeting Agent

The 5T4 targeting agents of the present invention are labeled with a radioisotope. According to certain aspects, the 5T4 targeting agent may be an antibody against 5T4 that is deglycosylated in the constant region, such as at asparagine-297 (Asn-297, N297; kabat number) in the heavy chain CH2 domain, for the purpose of uncovering a unique conjugation site, glutamine (i.e., Gln-295, Q295) so that it is available for conjugation with bifunctional chelator molecules.

According to certain aspects, the 5T4 targeting agent may be an antibody against that may have reduced disulfide bonds such as by using reducing agents, which may then be converted to dehydroalanine for the purpose of conjugating with a bifunctional chelator molecule.

According to certain aspects, the 5T4 targeting agent may be an antibody against that may have reduced disulfide bonds, such as by use of reducing agents, followed by conjugation via aryl bridges with a bifunctional chelator molecule. For example, according to certain aspects a linker molecule such as 3,5-bis(bromomethyl)benzene may bridge the free sulfhydryl groups on the 5T4 targeting agent.

According to certain aspects, the 5T4 targeting agent may be an antibody against that may have certain specific existing amino acids replaced with cysteine(s) that then can be used for site-specific labeling.

According to certain aspects, the 5T4 targeting agents may be radiolabeled through site-specific conjugation of suitable bifunctional chelators. Exemplary linker/chelator molecules that may be used include at least p-SCN-Bn-DOTA, NH₂-DOTA, NH₂—(CH₂)₁₋₂₀-DOTA, NH₂-(PEG)₁₋₂₀-DOTA, HS-DOTA, HS—(CH₂)₁₋₂₀-DOTA, HS-(PEG)₁₋₂₀-DOTA, dibromo-S—(CH₂)₁₋₂₀-DOTA, dibromo-S-(PEG)₁₋₂₀-DOTA, p-SCN-Bn-DOTP, NH₂-DOTP, NH₂—(CH₂)₁₋₂₀-DOTP, NH₂-(PEG)₁₋₂₀-DOTP, HS-DOTP, HS—(CH₂)₁₋₂₀-DOTP, HS-(PEG)₁₋₂₀-DOTP, dibromo-S—(CH₂)₁₋₂₀-DOTP, and dibromo-S-(PEG)₁₋₂₀-DOTP.

According to certain aspects, the chelator molecules may be attached to the 5T4 targeting agent through a linker molecule.

Protein 5T4-targeting agents (and other proteins targeting other targets), such as antibodies and antigen-binding antibody fragments, may, for example, be conjugated with a chelator for radiolabeling of the targeting agent via chelation of a radionuclide. Such protein targeting agents, for example, that include lysine(s), may conveniently be conjugated to a DOTA chelating moiety using the bifunctional agent S-2-(4-Isothiocyanatobenzyl)-1,4,7,10-tetraazacyclododecane tetraacetic acid a/k/a/ “p-SCN-Bn-DOTA” (Catalog #B205; Macrocyclics, Inc., Plano, TX, USA). p-SCN-Bn-DOTA may be synthesized by a multi-step organic synthesis fully described in U.S. Pat. No. 4,923,985. Chelation of a radionuclide by the DOTA moiety may be performed prior to chemical conjugation of the antibody with p-SCN-Bn-DOTA and/or after said conjugation.

Methods for conjugation and chelation of an exemplary radionuclide are further discussed in Example 1.

Diagnostics

The presently disclosed methods may include diagnosing the subject to ascertain if 5T4-positive cells are present. 5T4-positive cells may be present in a number of biological specimens, such as in circulating cells in a sample of blood from the subject or tumor cells in a biopsy of the subject. The diagnosing step may generally include obtaining a sample of blood or tissue from the subject and mounting the sample on a substrate. The presence or absence of the 5T4 antigen may be detected using a diagnostic antibody, peptide, or small molecule, wherein the diagnostic antibody peptide, or small molecule is labeled with any of the standard imaging labels known in the art. Exemplary labeling agents include that may be used, for example, radiolabels such as ³H, ¹⁴C, ³²P, ³⁵S, and ¹²⁵I; fluorescent or chemiluminescent compounds, such as fluorescein isothiocyanate, rhodamine, or luciferin; and enzymes, such as alkaline phosphatase, β-galactosidase, or horseradish peroxidase. An exemplary 5T4 targeting agent that may be used in such a diagnostic assay includes a human or humanized antibody against 5T4.

Alternatively, the methods may include diagnosing the subject to ascertain if 5T4-positive cells are present using a 5T4 targeting agent labeled with any of ¹⁸F, ¹¹C, ⁶⁸Ga, ⁶⁴Cu, ⁸⁹Zr or ¹²⁴I, which are useful for PET imaging, or ^{99m}Tc or ¹¹¹In, which are useful for SPECT imaging. Accordingly, the method may include administering to the subject a 5T4 targeting agent labeled with one or more of ¹⁸F, ¹¹C, ⁶⁸Ga, ⁶⁴Cu, ⁸⁹Zr, ¹²⁴I, ^{99m}Tc, or ¹¹¹In, and performing a non-invasive imaging technique on the subject, such as performing a PET or SPECT scan on the subject. The method may further include, before performing the imaging, waiting a sufficient amount of time for the 5T4 targeting agent to accumulate in tissues of the subject. According to certain preferred aspect of the method, the 5T4 targeting agent may include ⁶⁸Ga, ⁸⁹Zr, or ¹¹¹In, and may be labeled using any of the methods disclosed herein (e.g., such as disclosed in Example 1).

If the subject has 5T4-positive cells, the methods of the presently disclosed invention may be carried out, i.e., administration of a therapeutically effective amount of an 5T4 targeting agent, alone or in combination with one or more additional therapeutic agents.

Additional Agents and Treatments

The methods of the present invention, which include administration of a 5T4 targeting agent, may further include administration of one or more additional therapeutic agents and/or therapeutic treatments such as external beam radiation or brachytherapy. According to certain aspects, the additional agent(s) may be relevant for the disease or condition being treated. Such administration may be simultaneous, separate or sequential with the administration of the effective amount of the 5T4 targeting agent. For simultaneous administration, the agents may be administered as one composition, or as separate compositions, as appropriate.

Exemplary additional therapeutic agents that may be used include at least chemotherapeutic agents, small molecule therapeutics, anti-inflammatory agents, immunosuppressive agents, immunomodulatory agents, therapeutic antibodies, or a combination thereof.

According to certain aspects of the present invention, the one or more further therapeutic agents may include an antimyeloma agent, such as dexamethasone, doxorubicin, bortezomib, lenalidomide, prednisone, carmustine, etoposide, cisplatin, vincristine, cyclophosphamide, and thalidomide.

According to certain aspects of the present invention, the methods may further include administration of a cytokine such as granulocyte colony-stimulating factor (GCSF) after administration of the 5T4 targeting agent. The GCSF may be administered, for example, 7, 8, 9, 10, or 11 days after administration of the 5T4 targeting agent.

Further exemplary additional agents that may be used include immune checkpoint therapies and/or DDR inhibitors such as PARPi.

Without limitation, further agents and treatments that may be used in combination with or in conjunction with the radiolabeled 5T4 targeting agents of the invention include the following.

Chemotherapeutic Agents and Small Molecule Therapeutics

Exemplary chemotherapeutic agents that may be used include, but are not limited to, anti-neoplastic agents including alkylating agents including: nitrogen mustards, such as mechlorethamine, cyclophosphamide, ifosfamide, melphalan and chlorambucil; nitrosoureas, such as carmustine (BCNU), lomustine (CCNU), and semustine (methyl-CCNU); Temodal™ (temozolamide), ethylenimines/methylmelamine such as thriethylenemelamine (TEM), triethylene, thiophosphoramide (thiotepa), hexamethylmelamine (HMM, altretamine); alkyl sulfonates such as busulfan; triazines such as dacarbazine (DTIC); antimetabolites including folic acid analogs such as methotrexate and trimetrexate, pyrimidine analogs such as 5-fluorouracil (5FU), fluorodeoxyuridine, gemcitabine, cytosine arabinoside (AraC, cytarabine), 5-azacytidine, 2,2′-difluorodeoxycytidine, purine analogs such as 6-mercaptopurine, 6-thioguamne, azathioprine, T-deoxycoformycin (pentostatin), erythrohydroxynonyladenine (EHNA), fludarabine phosphate, and 2-chlorodeoxyadenosine (cladribine, 2-CdA); natural products including antimitotic drugs such as paclitaxel, vinca alkaloids including vinblastine (VLB), vincristine, and vinorelbine, taxotere, estramustine, and estramustine phosphate; pipodophylotoxins such as etoposide and teniposide; antibiotics such as actinomycin D, daunomycin (rubidomycin), doxorubicin, mitoxantrone, idarubicin, bleomycins, plicamycin (mithramycin), mitomycin C, and actinomycin; enzymes such as L-asparaginase; biological response modifiers such as interferon-alpha, IL-2, G-CSF and GM-CSF; miscellaneous agents including platinum coordination complexes such as oxaliplatin, cisplatin and carboplatin, anthracenediones such as mitoxantrone, substituted urea such as hydroxyurea, methylhydrazine derivatives including N-methylhydrazine (MIH) and procarbazine, adrenocortical suppressants such as mitotane (o, p-DDD) and aminoglutethimide; hormones and antagonists including adrenocorticosteroid antagonists such as prednisone and equivalents, dexamethasone and aminoglutethimide; Gemzar™ (gemcitabine), progestin such as hydroxyprogesterone caproate, medroxyprogesterone acetate and megestrol acetate; estrogen such as diethylstilbestrol and ethinyl estradiol equivalents; antiestrogen such as tamoxifen; androgens including testosterone propionate and fluoxymesterone/equivalents; antiandrogens such as flutamide, gonadotropin-releasing hormone analogs and leuprolide; and non-steroidal antiandrogens such as flutamide. Therapies targeting epigenetic mechanism including, but not limited to, histone deacetylase inhibitors, demethylating agents (e.g., Vidaza®) and release of transcriptional repression (ATRA) therapies can also be combined with antibodies of the invention.

According to certain aspects, the chemotherapeutic agent may be selected from the group consisting of taxanes (e.g., paclitaxel (Taxol), docetaxel (Taxotere), modified paclitaxel (e.g., Abraxane and Opaxio), doxorubicin, sunitinib (Sutent), sorafenib (Nexavar), and other multikinase inhibitors, oxaliplatin, cisplatin and carboplatin, etoposide, gemcitabine, and vinblastine. In one embodiment the chemotherapeutic agent is selected from the group consisting of taxanes (e.g. taxol (paclitaxel), docetaxel (Taxotere), modified paclitaxel (e.g. Abraxane and Opaxio)).

According to aspects of the presently disclosed invention, the chemotherapeutic agent is selected from 5-fluorouracil (5-FU), leucovorin, irinotecan, or oxaliplatin. According to certain aspects, the chemotherapeutic agent is 5-fluorouracil, leucovorin and irinotecan (FOLFIRI). According to other aspects, the chemotherapeutic agent is 5-fluorouracil, and oxaliplatin (FOLFOX).

According to aspects of the presently disclosed invention, the chemotherapeutic agent is selected from taxanes (e.g., docetaxel or paclitaxel) or a modified paclitaxel (e.g., Abraxane or Opaxio), doxorubicin), capecitabine and/or bevacizumab (Avastin) for the treatment of breast cancer; therapies with carboplatin, oxaliplatin, cisplatin, paclitaxel, doxorubicin (or modified doxorubicin (Caelyx or Doxil)), or topotecan (Hycamtin) for the treatment of ovarian cancer; therapies with a multi-kinase inhibitor, MKI, (Sutent, Nexavar, or 706) and/or doxorubicin for the treatment of kidney cancer; therapies with oxaliplatin, cisplatin and/or radiation for the treatment of squamous cell carcinoma; and therapies with taxol and/or carboplatin for the treatment of lung cancer.

The additional agents may, for example, include at least radiosensitizers, such as temozolomide, cisplatin, and/or fluorouracil.

The additional agents may, for example, include a bcl-2 inhibitor such as navitoclax or venetoclax (Venclexta®; Abbvie) and the combination may, for example, be used for the treatment of solid tumors such as breast cancers and lunger cancer such as small cell lung carcinoma (SCLC) as well as hematological malignancies including lymphomas and leukemias such as chronic lymphocytic leukemia (CLL), small lymphocytic lymphoma (SLL), and acute myeloid leukemia (AML).

The additional agents may, for example, include a cyclin-dependent kinase CDK4 and CDK6 inhibitor such as palbociclib (Ibrance®; Pfizer) and the combination may, for example, be used for the treatment of breast cancers such as HR-positive and HER2-negative breast cancer, with or without an aromatase inhibitor.

The additional agents may, for example, include erlotinib (Tarceva®; Roche) and the combination may, for example, be used for the treatment of solid tumor cancers such as non-small cell lung cancer (NSCLC), for example, with mutations in the epidermal growth factor receptor (EGFR) and pancreatic cancer.

The additional agents may, for example, include sirolimus or everolimus (Affinitor®; Novartis) and the combination may, for example, be used for the treatment of solid tumor cancers such as melanoma and breast cancer, or for hematological cancers such as lymphomas and lymphoblastic leukemias such as acute lymphoblastic leukemia.

The additional agents may, for example, include pemetrexed (Alimta®; Eli Lilly) and the combination may, for example, be used for the treatment of mesothelioma such as pleural mesothelioma and lung cancer such as non-small cell lung cancer (NSCLC).

5T4 has also been shown to be expressed in certain hematological malignancies. See Castro et al., 2012. 5T4 oncofoetal antigen is expressed in high risk of relapse childhood pre-B acute lymphoblastic leukemia and is associated with a more invasive and chemotactic phenotype, Leukemia. 2012 July; 26(7): 1487-1498.

The additional agents may, for example, include thalidomide or lenalidomide (Revlimid®; Celgene) and the combination may, for example, be used for the treatment of 5T4-expressing cancers such as solid tumors and hematological proliferative disorders, such as acute lymphoblastic leukemia (ALL), multiple myeloma, myelodysplastic syndromes, and mantle cell lymphoma.

The additional agents may, for example, include bortezomib (Velcade®; Takeda) and the combination may, for example, for the treatment of 5T4-expressing cancers such as hematological proliferative disorders, such as acute lymphoblastic leukemia (ALL), multiple myeloma, and mantle cell lymphoma.

The additional agents may, for example, include ibrutinib (Imbruvica®; Abbvie) and the combination may, for example, be used for the treatment of 5T4-expressing cancers such as solid tumors such as melanoma, gastrointestinal stromal tumors (GIST), and lung, breast, and prostate cancers, and hematological proliferative disorders, such as acute lymphoblastic leukemia (ALL), mantle cell lymphoma and chronic lymphocytic leukemia.

The additional agents may, for example, include nilotinib (Tasigna®; Novartis) and the combination may, for example, be used for the treatment of 5T4-expressing cancers such solid tumors and hematological proliferative disorders, such as acute lymphoblastic leukemia, such as Philadelphia chromosome-positive ALL, and chronic myelogenous leukemia, such as chronic myelogenous leukemia having the Philadelphia chromosome.

The additional agents may, for example, include imatinib (Gleevec®; Novartis) and the combination may, for example, be used for the treatment of 5T4-expressing cancers such as chronic myelogenous leukemia (CIVIL) and acute lymphocytic leukemia (ALL) such as those that are Philadelphia chromosome-positive (Ph+), gastrointestinal stromal tumors (GIST), hypereosinophilic syndrome (HES), chronic eosinophilic leukemia (CEL), systemic mastocytosis, and myelodysplastic syndrome.

The therapeutic agents may, for example, be administered according to any standard dose regime known in the field. Therapeutic agents may, for example, be administered at concentrations in the range of 1 to 500 mg/m², the amounts being calculated as a function of patient surface area (m²). Exemplary doses of the chemotherapeutic paclitaxel include 15 mg/m² to 275 mg/m², exemplary doses of docetaxel include 60 mg/m² to 100 mg/m², exemplary doses of epothilone include 10 mg/m² to 20 mg/m², and exemplary doses of calicheamicin include 1 mg/m² to 10 mg/m². While exemplary doses are listed herein, such are only provided for reference and are not intended to limit the dose ranges of the drug agents of the presently disclosed invention.

External Beam Radiation and/or Brachytherapy

The additional therapeutic modality used with the radiolabeled 5T4 targeting agent, and optionally any other of the other additional agents and/or therapies disclosed herein, may include an ionizing radiation administered, for example, via external beam radiation or brachytherapy. The radiation administered may, for example, include X-rays, gamma rays, or charged particles (e.g., protons or electrons) to generate ionizing radiation, such as delivered by a machine placed outside the patient's body (external-beam radiation therapy) or by a source placed inside a patient's body (internal radiation therapy or brachytherapy).

The external beam radiation or brachytherapy may enhance the targeted radiation damage delivered by the radiolabeled 5T4 targeting agent and may thus be delivered sequentially with the radiolabeled 5T4 targeting agent, such as before and/or after the radiolabeled 5T4 targeting agent, or simultaneous with the radiolabeled 5T4 targeting agents.

The external beam radiation or brachytherapy may, for example, be planned and administered in conjunction with imaging-based techniques such as computed tomography (CT) and/or magnetic resonance imaging (MRI) to accurately determine the dose and location of radiation to be administered. For example, a patient treated with any of the radiolabeled 5T4 targeting agents disclosed herein may be imaged using either of CT or MRI to determine the dose and location of radiation to be administered by the external beam radiation or brachytherapy.

The radiation therapy may, for example, be selected from the group consisting of total all-body radiation therapy, conventional external beam radiation therapy, stereotactic radiosurgery, stereotactic body radiation therapy, 3-D conformal radiation therapy, intensity-modulated radiation therapy, image-guided radiation therapy, tomotherapy, and brachytherapy. The radiation therapy may be provided as a single dose or as fractionated doses, e.g., as 2 or more fractions. For example, the dose may be administered such that each fraction includes 2-20 Gy (e.g., a radiation dose of 50 Gy may be split up into 10 fractions, each including 5 Gy). The 2 or more fractions may be administered on consecutive or sequential days, such as once in 2 days, once in 3 days, once in 4 days, once in 5 days, once in 6 days, once in 7 days, or in a combination thereof

Immune Checkpoint Therapies

The additional agent(s) used with the 5T4 targeting agent may include an immune checkpoint therapy. Cancer cells have developed means to evade the standard checkpoints of the immune system. For example, cancer cells have been found to evade immunosurveillance through reduced expression of tumor antigens, downregulation of MEW class I and II molecules leading to reduced tumor antigen presentation, secretion of immunosuppressive cytokines such as TGFb, recruitment or induction of immunosuppressive cells such as regulatory T cells (Treg) or myeloid-derived suppressor cells (MDSC), and overexpression of certain ligands [e.g., programmed death ligand-1 (PD-L1)] that inhibit the host's existing antitumor immunity.

Another major mechanism of immune suppression by cancer cells is a process known as “T-cell exhaustion”, which results from chronic exposure to tumor antigens, and is characterized by the upregulation of inhibitory receptors. These inhibitory receptors serve as immune checkpoints in order to prevent uncontrolled immune reactions.

Various immune checkpoints acting at different levels of T cell immunity have been described in the literature, including PD-1 (i.e., programmed cell death protein 1) and its ligands PD-L1 and PD-L2, CTLA-4 (i.e., cytotoxic T-lymphocyte associated protein-4) and its ligands CD80 and CD86, LAG3 (i.e., Lymphocyte-activation gene 3), B and T lymphocyte attenuator, TIGIT (T-cell immunoreceptor with Ig and ITIM domains), TIM-3 (i.e., T-cell immunoglobulin and mucin-domain containing protein 3), and VISTA (V-domain immunoglobulin suppressor of T cell activation).

Enhancing the efficacy of the immune system by therapeutic intervention is a particularly exciting development in cancer treatment. As indicated, checkpoint inhibitors such as CTLA-4 and PD-1 prevent autoimmunity and generally protect tissues from immune collateral damage. In addition, stimulatory checkpoints, such as OX40 (i.e., tumor necrosis factor receptor superfamily, member 4; TNFR-SF4), CD137 (i.e., TNFR-SF9), GITR (i.e., Glucocorticoid-Induced TNFR), CD27 (i.e., TNFR-SF7), CD40 (i.e., cluster of differentiation 40), and CD28, activate and/or promote the expansion of T-cells. Regulation of the immune system by inhibition or overexpression of these proteins is an area of promising current research.

Thus, a promising therapeutic strategy is the use of immune checkpoint therapies that may remove certain blockades on the immune system that are utilized by cancer cells, in combination with the 5T4 targeting agents disclosed herein. For example, antibodies against certain immune checkpoint inhibitors (ICI) may block interaction between checkpoint inhibitor proteins and their ligands, therefore preventing the signaling events that would otherwise have led to inhibition of an immune response against the tumor cell.

Moreover, there is a growing body of preclinical evidence supporting the ability of radiation to synergize with ICI antibodies, and this is also being explored in the clinic with increasing numbers of clinical trials evaluating the combination of external beam radiation with immune checkpoint therapies across various tumor types and ICI antibodies (Lamichhane, 2018). Clinical evidence supporting this combination has been generated in melanoma, with two studies demonstrating a clinical benefit using radiation in combination with the anti-cytotoxic T-lymphocyte-associated protein 4 (CTLA-4) ICI antibody, ipilmumab (Twyman-Saint Vistor, 2015).

Accordingly, an object of the presently disclosed invention is to provide therapies for the treatment of cancer using a 5T4 targeting agent in combination with one or more immune checkpoint therapies, such as an ICI antibody.

Immune checkpoint therapies of the present invention include molecules that totally or partially reduce, inhibit, interfere with or modulate one or more checkpoint proteins. Checkpoint proteins regulate T-cell activation or function. Immune checkpoint therapies may unblock an existing immune response inhibition by binding to or otherwise disabling checkpoint inhibition. The immune checkpoint therapies may include monoclonal antibodies, humanized antibodies, fully human antibodies, antibody fragments, small molecule therapeutics, or a combination thereof.

Exemplary immune checkpoint therapies that may be used may specifically bind to and inhibit a checkpoint protein, such as the inhibitory receptors CTLA-4, PD-1, TIM-3, VISTA, BTLA, LAG-3 and TIGIT, and/or the activating receptors CD28, OX40, CD40, GITR, CD137, CD27, and HVEM. Additionally, the immune checkpoint therapy may bind to a ligand of any of the aforementioned checkpoint proteins, such as PD-L1, PD-L2, PD-L3, and PD-L4 (ligands for PD-1); CD80 and CD86 (ligands for CTLA-4); CD137-L (ligand of CD137); and GITR-L (ligand of GITR). Other exemplary immune checkpoint therapies that may be used may bind to checkpoint proteins such as CD226, B7-H3, B7-H4, BTLA, TIGIT, GALS, KIR, 2B4 (belongs to the CD2 family of molecules and is expressed on all NK, γδ, and memory CD8+ (αβ) T cells), CD160 (also referred to as BY55), and CGEN-15049.

Central to the immune checkpoint process are the CD137, CTLA-4 and PD-1 immune checkpoint pathways.

The CTLA-4 and PD-1 pathways are thought to operate at different stages of an immune response. CTLA-4 is considered the “leader” of the immune checkpoint inhibitors (ICI), as it stops potentially autoreactive T cells at the initial stage of naive T-cell activation, typically in lymph nodes. The PD-1 pathway regulates previously activated T cells at the later stages of an immune response, primarily in peripheral tissues. Moreover, progressing cancer patients have been shown to lack upregulation of PD-L1 by either tumor cells or tumor-infiltrating immune cells. Immune checkpoint therapies targeting the PD-1 pathway might thus be especially effective in tumors where this immune suppressive axis is operational and reversing the balance towards an immune protective environment would rekindle and strengthen a pre-existing anti-tumor immune response. PD-1 blockade can be accomplished by a variety of mechanisms including antibodies that bind PD-1 or its ligand, PD-L1.

According to certain aspects of the presently disclosed invention, the immune checkpoint therapy may include an inhibitor of the PD-1 checkpoint, which may decrease, block, inhibit, abrogate, or interfere with signal transduction resulting from the interaction of PD-1 with one or more of its binding partners, such as PD-L1 and PD-L2. The inhibitor of the PD-1 checkpoint may be an anti-PD-1 antibody, antigen binding fragment, fusion proteins, oligopeptides, and other molecules that decrease, block, inhibit, abrogate or interfere with signal transduction resulting from the interaction of PD-1 with PD-L1 and/or PD-L2. In some embodiments, a PD-1 checkpoint inhibitor reduces the negative co-stimulatory signal mediated by or through cell surface proteins expressed on T lymphocytes so as render a dysfunctional T-cell less dysfunctional (e.g., enhancing effector responses to antigen recognition). In some embodiments, the PD-1 checkpoint therapy is an anti-PD-1 antibody.

Thus, according to certain aspects of the present invention, the immune checkpoint therapy may include a monoclonal antibody against an immune checkpoint inhibitor (ICI) such as against CTLA-4, PD-1, or PD-L1.

According to certain aspects, the ICI antibody may be an antibody against PD-1. The ICI antibody may be an anti-PD-1 antibody, such as nivolumab. For example, the inhibitors of PD-1 biological activity (or its ligands) disclosed in U.S. Pat. No. 7,029,674. Exemplary antibodies against PD-1 that may be used include: Anti-mouse PD-1 antibody Clone J43 (Cat #BE0033-2) from BioXcell; Anti-mouse PD-1 antibody Clone RMP1-14 (Cat #BE0146) from BioXcell; mouse anti-PD-1 antibody Clone EH12; Merck's MK-3475 anti-mouse PD-1 antibody (Keytruda®, pembrolizumab, lambrolizumab); and AnaptysBio's anti-PD-1 antibody, known as ANB011; antibody MDX-1 106 (ONO-4538); Bristol-Myers Squibb's human IgG4 monoclonal antibody nivolumab (Opdivo®, BMS-936558, MDX1106); Regeneron's monoclonal antibody cemiplimab (Libtayo®), AstraZeneca's AMP-514, and AMP-224; and Pidilizumab (CT-011), CureTech Ltd.

According to certain aspects, the immune checkpoint therapy is an inhibitor of PD-L1. Exemplary inhibitors of PD-L1 that may be used include antibodies (e.g., an anti-PD-L1 antibody, i.e., ICI antibody), RNAi molecules (e.g., anti-PD-L1 RNAi), antisense molecules (e.g., an anti-PD-L1 antisense RNA), dominant negative proteins (e.g., a dominant negative PD-L1 protein), and small molecule inhibitors. An exemplary anti-PD-L1 antibody that may be used includes clone EH12. Exemplary antibodies against PD-L1 that may be used include: Genentech's MPDL3280A (RG7446); anti-mouse PD-L1 antibody Clone 10F.9G2 (Cat #BE0101) from BioXcell; anti-PD-L1 monoclonal antibody MDX-1105 (BMS-936559) and BMS-935559 from Bristol-Meyer's Squibb; MSB0010718C; mouse anti-PD-L1 Clone 29E.2A3; and AstraZeneca's MEDI4736 (Durvalumab; Imfinzi®).

According to certain aspects, the immune checkpoint therapy is an inhibitor of PD-L2 or may reduce the interaction between PD-1 and PD-L2. Exemplary inhibitors of PD-L2 that may be used include antibodies (e.g., an anti-PD-L2 antibody, i.e., ICI antibody), RNAi molecules (e.g., an anti-PD-L2 RNAi), antisense molecules (e.g., an anti-PD-L2 antisense RNA), dominant negative proteins (e.g., a dominant negative PD-L2 protein), and small molecule inhibitors. Antibodies include monoclonal antibodies, humanized antibodies, deimmunized antibodies, and Ig fusion proteins.

According to certain aspects, the immune checkpoint therapy may be an inhibitor of CTLA-4, such as an anti-CTLA-4 antibody, i.e., ICI antibody. According to one aspect, the ICI antibody may be ipilimumab. The anti-CTLA-4 antibody may block the binding of CTLA-4 to CD80 (B7-1) and/or CD86 (B7-2) expressed on antigen presenting cells. Exemplary antibodies against CTLA-4 that may be used include: Bristol Meyers Squibb's anti-CTLA-4 antibody ipilimumab (also known as Yervoy®, MDX-010, BMS-734016 and MDX-101); anti-CTLA4 Antibody, clone 9H10 from Millipore; Pfizer's tremelimumab (CP-675,206, ticilimumab); and anti-CTLA-4 antibody clone BNI3 from Abcam. According to certain aspects, the immune checkpoint inhibitor may be a nucleic acid inhibitor of CTLA-4 expression.

CD137 (also known “TNF receptor superfamily member 9”) is a costimulatory receptor member of the tumor necrosis factor receptor superfamily, mediating CD28-dependent and independent T-cell co-stimulation (Bartkowiak, 2015). CD137 is inducibly expressed by T cells, natural killer (NK) cells, dendritic cells (DC), B cells, and other cells of the immune system. The protein is composed of a 255-amino acid protein having a short N-terminal cytoplasmic portion, a transmembrane region, and an extracellular domain that possesses 3 cysteine-rich motifs. Ligation of CD137 by its ligand CD137L (4-1BBL; TNFSF9), which is mainly, though not exclusively, expressed on Antigen-Presenting Cells (APCs), evokes various T cell responses such as cell expansion, increased cytokine secretion and the prevention of activation-induced cell death. Thus, such ligation serves to activate the immune system. However, cis-interactions between CD137 and CD137L also potently downregulate the expression of CD137L (Kwon, 2015). The CD137 ligand thus functions to control the extent and kinetics of CD137-mediated immune system activation (Kwon, 2015). Significantly, CD137 expressed on human NK cells becomes upregulated upon binding to anti-tumor antibodies that have become bound to tumor cells (Wei, 2014).

Thus, according to certain aspects of the presently disclosed invention, the immune checkpoint therapy may include an antibody against CD137, which could be used to activate the immune system and thereby provide a therapy for cancer in combination with the presently disclosed 5T4 targeting agents. Exemplary anti-CD137 antibodies that may be used are disclosed in U.S. Pat. Nos. 2014/0274909; 2013/0280265; 2013/0273078; 2013/0071403; 2012/0058047; 2011/0104049; 2011/0097313; 2008/0166336; 2008/0019905; 2006/0188439; 2006/0182744; 2006/0121030; and 2003/0223989.

According to certain aspects of the present invention, the immune checkpoint therapy may include more than one modulator of an immune checkpoint protein. As such, the immune checkpoint therapy may include a first antibody or inhibitor against a first immune checkpoint protein and a second antibody or inhibitor against a second immune checkpoint protein.

CD47 Blockades

The additional agents used with the radiolabeled 5T4 targeting agent may include a CD47 blockade, such as any agent that interferes with, or reduces the activity and/or signaling between CD47 (e.g., on a target cell) and SIRPα (e.g., on a phagocytic cell) through interaction with either CD47 or SIRPα. Non-limiting examples of suitable CD47 blockades include CD47 and/or SIRPα reagents, including without limitation SIRPα polypeptides, anti-SIRPα antibodies, soluble CD47 polypeptides, and anti-CD47 antibodies or antibody fragments.

Additional examples of a CD47 blockade include agents that modulate the expression of CD47 and/or SIRPα. For example, such agents may include nucleic acid approaches such as antisense phosphorodiamidate morpholino oligomers (PMO) that block translation of CD47 or antibodies specific for human CD47 that modulate, e.g., block, inhibit, reduce, antagonize, neutralize or otherwise interfere with CD47 expression.

The anti-CD47 agent may, for example, be an antibody that specifically binds CD47 (i.e., an anti-CD47 antibody) and reduces the interaction between CD47 on one cell (e.g., an infected cell) and SIRPα on another cell (e.g., a phagocytic cell). Non-limiting examples of suitable antibodies include clones B6H12, 5F9, 8B6, and C3 (for example as described in International Patent Publication WO 2011/143624). Suitable anti-CD47 antibodies include fully human, humanized or chimeric versions of such antibodies.

TABLE 1

| Company | Approach | Program |
|---|---|---|
| Akesobio Australia Pty Ltd | CD47 mAb | AK117 |
| Arch Oncology (Tioma | CD47 mAb | AO-176 |
| Therapeutics) | | |
| Elpiscience Biopharma Inc. | CD47 | ES004 |
| EpicentRx | Small molecule inhibitor of | RRx-001 |
| | dinitroazetidine hypoxia sensor | (1-bromoacetyl-3,3 |
| | to downregulate CD47/SIRPα | dinitroazetidine) |
| ImmuneOncia Therapeutics | CD47 mAb human | IMC-002 |
| Innovent Biologics | CD47 mAb | IBI-188 (CD47 mAb) |
| | CD47/PD-L1 bispecific mAb | IBI-322 (Bispecific) |
| OSE | SIRPα mAb | BI 765063 (OSE-172) |
| Zai Lab | CD47 mAb | ZL-1201 |
| Alx Oncology | High-affinity SIRPα-Fc | ALX148 |
| Gilead/Forty Seven | CD47 mAb | Magrolimab FSI-189 |
| | SIRPα mAb | |
| I-Mab Biopharma | CD47 mAb | TJC4 |
| Jiangsu HengRui Medicine | CD47 mAb | SHR-1603 |
| Co., Ltd. | | |
| Surface Oncology | CD47 mAb human | SRF231 |
| Morphiex | CD47 targeting | MBT-001 |
| | phosphorodiamidate morpholino | |
| | oligomers | |

Exemplary human or humanized antibodies especially useful for in vivo applications in humans due to their low antigenicity include at least monoclonal antibodies against CD47, such as Hu5F9-G4, a humanized monoclonal antibody available from Gilead as Magrolimab (Sikic, et al. (2019) Journal of Clinical Oncology 37:946); Lemzoparlimab and TJC4 from I-Mab Biopharma; AO-176 from Arch Oncology, Inc; AK117 from Akesobio Australia Pty; IMC-002 from Innovent Biologics; ZL-1201 from Zia Lab; SHR-1603 from Jiangsu HengRui Medincine Co.; and SRF231 from Surface Oncology. Bispecific monoclonal antibodies are also available, such as IBI-322, targeting both CD47 and PD-L1 from Innovent Biologics. Antibodies against SIRPα are also possible, such as ALX148 from Alx Oncology; BI 765063 (OSE-172) from OSE; as well as small molecule inhibitors, such as RRx-001 from EpicentRx, that is an inhibitor of dinitroazetidine hypoxia sensor to downregulate CD47/SIRPα (see Table 1).

AO-176, in addition to inducing tumor phagocytosis through blocking the CD47-SIRPα interaction, has been found to preferentially bind tumor cells versus normal cells (particularly RBCs where binding is negligible) and directly kills tumor versus normal cells.

Other CD47 blockades that may be employed include any of those disclosed in U.S. Pat. No. 9,969,789 including without limitation the SIRPα-IgG Fc fusion proteins TTI-621 and TTI-622 (Trillium Therapeutics, Inc.), both of which preferentially bind CD47 on tumor cells while also engaging activating Fc receptors.

The CD47 blockade may, for example, include a soluble CD47 polypeptide that specifically binds SIRPα and reduces the interaction between CD47 on one cell (e.g., an infected cell) and SIRPα on another cell (e.g., a phagocytic cell). A suitable soluble CD47 polypeptide can bind SIRPα without activating or stimulating signaling through SIRPα because activation of SIRPα would inhibit phagocytosis. Instead, suitable soluble CD47 polypeptides facilitate the preferential phagocytosis of infected cells over non-infected cells. Those cells that express higher levels of CD47 (e.g., infected cells) relative to normal, non-target cells (normal cells) will be preferentially phagocytosed. Thus, a suitable soluble CD47 polypeptide specifically binds SIRPα without activating/stimulating enough of a signaling response to inhibit phagocytosis. A suitable soluble CD47 polypeptide may, for example, be a fusion protein such as any of those disclosed in U.S. Pub. No. 20100239579.

Therapeutically effective doses of an anti-CD47 antibody or other blocking agent may, for example, be a dose that leads to sustained serum levels of the anti-CD47 antibody of about 40 μg/ml or more (e.g., about 50 ug/ml or more, about 60 ug/ml or more, about 75 ug/ml or more, about 100 ug/ml or more, about 125 ug/ml or more, or about 150 ug/ml or more). Therapeutically effective doses may, for example, include administration of the anti-CD47 antibody in amounts of 0.05-10 mg/kg, such as at least 0.1 mg/kg, 0.5 mg/kg, 1.0 mg/kg, 1.5 mg/kg, 2.0 mg/kg, 2.5 mg/kg, 3.0 mg/kg, 3.5 mg/kg, 4.0 mg/kg, 4.5 mg/kg, 5.0 mg/kg, 5.5 mg/kg, 6.0 mg/kg, 6.5 mg/kg, 7.0 mg/kg, 7.5 mg/kg, 8.0 mg/kg, 8.5 mg/kg, 9.0 mg/kg; or not more than 10 mg/kg, 9.5 mg/kg, 9.0 mg/kg, 8.5 mg/kg, 8.0 mg/kg, 7.5 mg/kg, 7.0 mg/kg, 6.5 mg/kg, 6.0 mg/kg, 5.5 mg/kg, 5.0 mg/kg, 4.5 mg/kg, 4.0 mg/kg, 3.5 mg/kg, 3.0 mg/kg, 2.5 mg/kg, 2.0 mg/kg, 1.5 mg/kg, 1.0 mg/kg, or any combination of these upper and lower limits.

DNA Damage Response Inhibitors

The additional agents used with the 5T4 targeting agent may be a DNA damage response inhibitor (DDRi). DNA damage can be due to endogenous factors, such as spontaneous or enzymatic reactions, chemical reactions, or errors in replication, or may be due to exogenous factors, such as UV or ionizing radiation or genotoxic chemicals. The repair pathways that overcome this damage are collectively referred to as the DNA damage response or DDR. This signaling network acts to detect and orchestrate a cell's response to certain forms of DNA damage, most notably double strand breaks and replication stress. Following treatment with many types of DNA damaging drugs and ionizing radiation, cells are reliant on the DDR for survival. It has been shown that disruption of the DDR can increase cancer cell sensitivity to these DNA damaging agents and thus may improve patient responses to such therapies.

Within the DDR, there are several DNA repair mechanisms, including base excision repair, nucleotide excision repair, mismatch repair, homologous recombinant repair, and non-homologous end joining. Approximately 450 human DDR genes code for proteins with roles in physiological processes. Dysregulation of DDR leads to a variety of disorders, including genetic, neurodegenerative, immune, cardiovascular, and metabolic diseases or disorders and cancers. For example, the genes OGG1 and XRCC1 are part of the base excision repair mechanism of DDR, and mutations in these genes are found in renal, breast, and lung cancers, while the genes BRCA1 and BRCA2 are involved in homologous recombination repair mechanisms and mutations in these genes leads to an increased risk of breast, ovarian, prostate, pancreatic, as well as gastrointestinal and hematological cancers, and melanoma. Exemplary DDR genes are provided in Table 2.

TABLE 2

| DNA repair | Gene | |
|---|---|---|
| mechanism | examples | Cancer |
| Base Excision | OGG1 | Renal, breast and lung cancer |
| Repair | XRCC1 | Non-small cell lung cancer |
| Nucleotide | ERCC1 | Lung and skin cancer, and glioma |
| Excision Repair | XP | Xeroderma pigmentosum predisposing to skin cancer. |
| | | Also increased risk of bladder and lung cancer |
| Mismatch Repair | MSH2, | Lynch syndrome predisposing to colorectal cancer as well |
| | MLH1 | as endometrial, ovarian, stomach, small intestine, |
| | | hepatobiliary tract, upper urinary tract, brain and skin |
| | | cancer |
| Homologous | BRCA1, | Increased risk of breast, ovarian, prostate, pancreatic, as |
| Recombinant | BRCA2 | well as gastrointestinal and hematological cancer, and |
| Repair | | melanoma |
| Non-homologous | KU70 | Breast, colorectal and lung cancer |
| End Joining | KU80 | Lung cancer |
| Cell cycle | ATM | Ataxia-telangiectasia predisposing to leukemia, breast and |
| checkpoints | | pancreatic cancer |
| | ATR | Leukemia, lymphoma, gastric and endometrial cancer |

An object of the presently disclosed invention is to administer radiolabeled 5T4 targeting agents that deliver ionizing radiation in combination with a DDRi. Thus, according to certain aspects, the additional agent(s) administered with the 5T4 targeting agent may target proteins in the DDR, i.e., DDR inhibitors or DDRi, thus maximizing DNA damage or inhibiting the repair if the damage, such as in G1 and S-phase and/or preventing repair in G2, ensuring the maximum amount of DNA damage is taken into mitosis, leading to cell death.

Moreover, one or more DDR pathways may be targeted to ensure cell death, i.e., lethality to the targeted cancer cells. For example, mutations in the BRCA1 and 2 genes alone may not be sufficient to ensure cell death, as other pathways, such as the PARP1 base excision pathway, may act to repair the DNA damage. Thus, combinations of multiple DDRi inhibitors or combining DDRi with antiangiogenic agents or immune checkpoint inhibitors, such as listed hereinabove, are possible and an object of the presently disclosed invention.

Exemplary DDRi—ATM and ATR Inhibitors

Ataxia telangiectasia mutated (ATM) and Ataxia telangiectasia mutated and Rad-3 related (ATR) are members of the phosphatidylinositol 3-kinase-related kinase (PIKK) family of serine/threonine protein kinases.

ATM is a serine/threonine protein kinase that is recruited and activated by DNA double-strand breaks. The ATM phosphorylates several key proteins that initiate activation of a DNA damage checkpoint, leading to cell cycle arrest, DNA repair, or cellular apoptosis. Several of these targets, including p53, CHK2, and H2AX, are tumor suppressors. The protein is named for the disorder ataxia telangiectasia caused by mutations of the ATM. The ATM belongs to the superfamily of phosphatidylinositol 3-kinase-related kinases (PIKKs), which includes six serine/threonine protein kinases that show a sequence similarity to a phosphatidylinositol 3-kinase (PI3K).

Like ATM, ATR is one of the central kinases involved in the DDR. ATR is activated by single stranded DNA structures, which may for example arise at resected DNA DSBs or stalled replication forks. When DNA polymerases stall during DNA replication, the replicative helicases continue to unwind the DNA ahead of the replication fork, leading to the generation of long stretches of single stranded DNA (ssDNA).

ATM has been found to assist cancer cells by providing resistance against chemotherapeutic agents and thus favors tumor growth and survival. Inhibition of ATM and/or ATR may markedly increase cancer cell sensitivity to DNA damaging agents, such as the ionizing radiation provided by the radiolabeled 5T4 targeting agent. Accordingly, an object of the presently disclosed invention includes administration of an inhibitor of ATM (ATMi) and/or ATR (ATRi), in combination with the 5T4 targeting agents, to inhibit or kill cancer cells, such as those expressing tor overexpressing 5T4.

The inhibitor of ATM (ATMi) or ATR (ATRi) may be an antibody, peptide, or small molecule that targets ATM or ATR, respectively. Alternatively, an ATMi or ATRi may reduce or eliminate activation of ATM or ATR by one or more signaling molecules, proteins, or other compounds, or can result in the reduction or elimination of ATM or ATR activation by all signaling molecules, proteins, or other compounds. ATMi and/or ATRi also include compounds that inhibit their expression (e.g., compounds that inhibit ATM or ATR transcription or translation). An exemplary ATMi KU-55933 that may be used suppresses cell proliferation and induces apoptosis. Other exemplary ATMi that may be used include at least KU-59403, wortmannin, CP466722, and KU-60019. Exemplary ATRi include at least Schisandrin B, NU6027, NVP-BEA235, VE-821, VE-822, AZ20, and AZD6738.

Exemplary DDRi—Wee1 Inhibitors

The checkpoint kinase Wee1 catalyzes an inhibitory phosphorylation of both CDK1 (CDC2) and CDK2 on tyrosine 15, thus arresting the cell cycle in response to extrinsically induced DNA damage. Deregulated Wee1 expression or activity is believed to be a hallmark of pathology in several types of cancer. For example, Wee1 is often overexpressed in glioblastomas, malignant melanoma, hepatocellular carcinoma, breast cancer, colon carcinoma, lung carcinoma, and head and neck squamous cell carcinoma. Advanced tumors with an increased level of genomic instability may require functional checkpoints to allow for repair of such lethal DNA damage. As such, the present inventors believe that Wee1 represents an attractive target in advanced tumors where its inhibition is believed to result in irreparable DNA damage. Accordingly, an object of the presently disclosed invention includes administration of an inhibitor of Wee1, in combination with the 5T4 targeting agents, to inhibit or kill cancer cells, such as those expressing tor overexpressing 5T4.

A Wee1 inhibitor may be an antibody, peptide, or small molecule that targets Wee1. Alternatively, a Wee1 inhibitor may reduce or eliminate Wee1 activation by one or more signaling molecules, proteins, or other compounds, or can result in the reduction or elimination of Wee1 activation by all signaling molecules, proteins, or other compounds. The term also includes compounds that decrease or eliminate the activation or deactivation of one or more proteins or cell signaling components by Wee1 (e.g., a Wee1 inhibitor can decrease or eliminate Wee1-dependent inactivation of cyclin and Cdk activity). Wee1 inhibitors also include compounds that inhibit Wee1 expression (e.g., compounds that inhibit Wee1 transcription or translation).

Exemplary Wee1 inhibitors that may be used include AZD-1775 (i.e., adavosertib), and inhibitors such as those described in, e.g., U.S. Pat. Nos. 7,834,019; 7,935,708; 8,288,396; 8,436,004; 8,710,065; 8,716,297; 8,791,125; 8,796,289; 9,051,327; 9,181,239; 9,714,244; 9,718,821; and 9,850,247; U.S. Pat. App. Pub. Nos. U.S. 2010/0113445 and 2016/0222459; and International Pub. Nos. WO 2002/090360, WO 2015/019037, WO 2017/013436, WO 2017/216559, WO 2018/011569, and WO 2018/011570.

Further Wee1 inhibitors that may be used include a pyrazolopyrimidine derivative, a pyridopyrimidine, 4-(2-chlorophenyl)-9-hydroxypyrrolo[3,4-c]carbazole-1,3-(2H, 6H)-dione (CAS No. 622855-37-2), 6-butyl-4-(2-chlorophenyl)-9-hydroxypyrrolo[3,4-c]carbazole-1,3-(2H,6H)-dione (CAS No. 62285550-9), 4-(2-phenyl)-9-hydroxypyrrolo[3,4-c]carbazole-1,3-(2H,6H)-dione (CAS No. 1177150-89-8), and an anti-Wee1 small interfering RNA (siRNA) molecule.

Exemplary DDRi—PARP Inhibitors

Another exemplary DDRi that may be used is an inhibitor of poly(ADP-ribose) polymerase (“PARP”). Inhibitors of the DNA repair protein PARP, referred to individually and collectively as “PARPi”, have been approved for use in a range of solid tumors, such as breast and ovarian cancer, particularly in patients having BRCA1/2 mutations. BRCA1 and 2 function in homologous recombination repair (HRR). When mutated, they induce genomic instability by shifting the DNA repair process from conservative and precise HRR to non-fidelitous methods such as DNA endjoining, which can produce mutations via deletions and insertions.

PARPi have been shown to exhibit synthetic lethality, as exhibited by potent single agent activity, in BRCA1/2 mutant cells. This essentially blocks repair of single-strand DNA breaks. Since HRR is not functional in these tumor cells, cell death results. Because most tumors do not carry BRCA1 or BRCA2 mutations, the potency of PARPi in such tumors is far less pronounced.

To date, the FDA has approved four PARPi drugs (olaparib, niraparib, rucaparib and talazoparib) as monotherapy agents, specifically in patients with germline and somatic mutations in the BRCA1 and BRCA2 genes. Along with veliparib, olaparib, niraparib and rucaparib were among the first generation of PARPi that entered clinical trials. Their IC50 values were found to be in the nanomolar range. In contrast, second generation PARPi like talazoparib have IC50 values in the picomolar range.

These PARPi all bind to the binding site of the cofactor, b nicotinamide adenine dinucleotide (b-NAD+), in the catalytic domain of PARP1 and PARP2. The PARP family of enzymes use NAD+ to covalently add Poly(ADP-ribose) (PAR) chains onto target proteins, a process termed “PARylation.” PARP1 (which is the best-studied member) and PARP2, are important components of the DNA damage response (DDR) pathway. PARP1 is involved in the repair of single-stranded DNA breaks, and possibly other DNA lesions (Woodhouse, et al.; Krishnakumar, et al.). Through its zinc finger domains, PARP1 binds to damaged DNA and then PARylates a series of DNA repair effector proteins, releasing nicotinamide as a by-product (Krishnakumar, et al.). Subsequently, PARP1 auto-PARylation leads to release of the protein from the DNA. The available PARPi, however, differ in their capability to trap PARP1 on DNA, which seems to correlate with cytotoxicity and drug efficacy. Specifically, drugs like talazoparib and olaparib are more effective in trapping PARP1 than are veliparib (Murai, et al., 2012; Murai, et al., 2014).

The efficacy of PARPi in ovarian cancer and breast cancer patients who have loss-of-function mutations in BRCA1 or BRCA2 genes is largely attributed to the genetic concept of synthetic lethality: that proteins of BRCA 1 and 2 normally maintain the integrity of the genome by mediating a DNA repair process, known as homologous recombination repair (HRR); and PARPi causes a persistent DNA lesion that, normally, would otherwise be repaired by HR. In the presence of PARPi, PARP1 is trapped on DNA which stalls progression of the replication fork. This stalling is cytotoxic unless timely repaired by the HR system. In cells lacking effective HR, they are unable to effectively repair these DNA lesions, and thus die.

Again, mutations in BRCA genes and others in the HRR system are not prevalent in many cancer types. So, to better harness the therapeutic benefits of PARPi in such cancers, one can induce “artificial” synthetic lethality by pairing a PARPi with either chemotherapy or radiation therapy. Preclinical studies have demonstrated that combining radiation therapy and PARPi can increase the sensitivity of BRCA1/2 mutant tumor cells to PARP inhibition and extend the sensitivity of non-mutant BRCA tumors to PARP inhibition. Additional studies have shown that ionizing radiation (IR) itself can mediate PARPi synthetic lethality in tumor cells.

Accordingly, an object of the presently disclosed invention is to administer radiolabeled 5T4 targeting agents that deliver ionizing radiation in combination with a PARPi.

In the various embodiments of this invention, the PARPi may be any known agent performing that function, and preferably, one approved by the FDA. For example, the PARPi is olaparib (Lynparza®), niraparib (Zejula®), rucaparib (Rubraca®) or talazoparib (Talzenna®).

Clinically, therapy with PARPi has resulted in sustained anti-tumor responses in a range of cancers including ovarian, prostate, pancreatic, and triple-negative breast cancers (TNBC). In one clinical trial, TNBC patients with germline BRCA1/2 mutations were treated with the PARPi, olaparib. While this therapy demonstrated a higher disease stabilization rate in BRCA1/2-mutant compared to non-mutant patients, there were no sustained responses achieved in either cohort (Gelmon, 2011).

The present inventors realized that the effect of PARPi may be improved through increases in dsDNA breaks induced by ionizing radiation provided by a 5T4 targeting agent while these repair pathways are being blocked by the PARPi. Exemplary PARPi that may be used include olaparib, niraparib, rucaparib and talazoparib.

Other Additional Agents

In a still further aspect of the invention, the additional agents may, for example, include an anti-VEGF monoclonal antibody such as bevacizumab (Avastin®; Roche) and the combination may, for example, be used for the treatment of colorectal cancer, lung cancer, breast cancer, renal cancers such as renal-cell carcinoma, brain cancers such as glioblastoma, ovarian cancer, and cervical cancer.

### EXAMPLES

### Example 1: Production of Radiolabeled 5T4 Targeting Agent

The 5T4 targeting agent, such as a monoclonal antibody against 5T4, may be labeled with Indium-111 (¹¹¹In) or Actinium-225 (²²⁵Ac) according to procedures detailed in any of U.S. Pat. No. 10,420,851, International Publication No. WO 2017/155937 and U.S. Provisional Patent Application No. 63/042,651 filed Dec. 9, 2019 and titled “Compositions and methods for preparation of site-specific radioconjugates.”

Radiolabeling: The antibody may be conjugated to a chelator, such as described hereinabove, and in the above referenced patent applications. An exemplary chelator that may be used includes at least dodecane tetraacetic acid (DOTA), wherein a goal of the conjugation reaction is to achieve a DOTA-antibody ratio of 3:1 to 5:1. Chelation with the radionuclide ¹¹¹In or ²²⁵Ac may then be performed and efficiency and purity of the resulting ¹¹¹In- or ²²⁵Ac-labeled anti-5T4 antibody may be determined by HPLC and iTLC.

An exemplary chelation labeling reaction for ²²⁵Ac is as follows: A reaction including 15 μl 0.15M NH₄OAc buffer, pH=6.5 and 2 μL (10 μg) DOTA-anti-5T4 (5 mg/ml) may be mixed in an Eppendorf reaction tube, and 4 μL ²²⁵Ac (10 μCi) in 0.05 M HCl subsequently added. The contents of the tube may be mixed with a pipette tip and the reaction mixture incubated at 37° C. for 90 min with shaking at 100 rpm. At the end of the incubation period, 3 μL of a 1 mM DTPA solution may be added to the reaction mixture and incubated at room temperature for 20 min to bind the unreacted ²²⁵Ac into the ²²⁵Ac-DTPA complex. Instant thin layer chromatography with 10 cm silica gel strip and 10 mM EDTA/normal saline mobile phase may be used to determine the radiochemical purity of ²²⁵Ac-DOTA-anti-5T4 through separating ²²⁵Ac-labeled anti-5T4 (²²⁵Ac-DOTA-anti-5T4) from free ²²⁵Ac (²²⁵Ac-DTPA). In this system, the radiolabeled antibody stays at the point of application and ²²⁵Ac-DTPA moves with the solvent front. The strips may be cut in halves and counted in the gamma counter equipped with the multichannel analyzer using channels 72-110 for ²²⁵Ac to exclude its daughters.

Purification: An exemplary radiolabeled 5T4 targeting agent, such as ²²⁵Ac-DOTA-anti-5T4, may be purified either on PD10 columns pre-blocked with 1% HSA or on Vivaspin centrifugal concentrators with a 50 kDa MW cut-off with 2×1.5 mL washes, 3 min per spin. HPLC analyses of the ²²⁵Ac-DOTA-anti-5T4 after purification may be conducted using a Waters HPLC system equipped with flow-through Waters UV and Bioscan Radiation detectors, using a TSK3000SW XL column eluted with PBS at pH=7.4 and a flow rate of 1 ml/min.

Stability determination: An exemplary radiolabeled 5T4 targeting agent, such as ²²⁵Ac-DOTA-anti-5T4, may be used for stability determination, wherein the ²²⁵Ac-DOTA-anti-5T4 may be tested either in the original volume or diluted (2- to 10-fold) with the working buffer (0.15 M NH₄OAc) and incubated at room temperature (rt) for 48 hours or at 4° C. for 96 hours and tested by ITLC. Stability is determined by comparison of the intact radiolabeled anti-5T4 before and after incubation. Other antibodies labeled with ²²⁵Ac have been found to be stable at 4° C. for up to 96 hrs.

Immunoreactivity (IR) determination: An exemplary radiolabeled 5T4 targeting agent, such as ²²⁵Ac-DOTA-anti-5T4, may be used in immunoreactivity experiments. 5T4 positive cells and control 5T4 negative cells may be used in the amounts of 1.0-7.5 million cells per sample to investigate the amount of binding (percent radioactivity binding to cells after several washes; or using an immunoreactive fraction (IRF) bead assay may be performed according to methods disclosed in as described by Sharma, 2019). Prior assays for other antibodies radiolabeled with ¹¹¹In or ²²⁵Ac demonstrated about 50-60% immunoreactivity.

### Example 2: Syngeneic Mouse Model

A syngeneic mouse model may be used to examine targeting efficiency and therapeutic efficacy of 5T4 targeting agents Such a model provides the opportunity to observe any toxicities that may arise through targeting this protein with a radioisotope warhead. It also allows for combination experiments with immune-modifying agents such an immune checkpoint inhibitor antibodies (PD-1, PD-L1, etc.).

Woods (2002) reported discovery of an antibody (9A7) that is reactive to mouse 5T4 and was used to screen mouse tumor lines for 5T4 expression (see Table 3; taken from Woods, 2002). Of the cell lines reported to be positive for 5T4 expression, the EMT6 mammary adenocarcinoma cell line has high levels of 5T4 expression, is readily available for purchase from commercial sources to perform experiments. Moreover, this cell line has been reported to be sensitive to radiation (Barnes, 2008).

TABLE 3

| Cell line | Origin | Flow cytometry |
|---|---|---|
| A9 neo | Lung fibroblast L cells | − |
| A9-m5T4 | Lung fibroblast L colls | ++++ |
| B16 F10 Neo | Melanoma | − |
| B16 F10-m5T4 | Melanoma | ++ |
| EMT6 | Mammary adrenocarcinoma | +++ |
| C1271 | Mammary carcinoma | +++ |
| Clone M3 | Melanoma | − |
| EL4 | Lymphoma | − |
| KLN-205 | Squamous cell lung carcinoma | +/− |
| JC | Breast adenocarcinoma | − |
| LL/2 | C57BL Lewis lung carcinoma | − |
| Mosec | Ovarian carcinoma | − |
| Nulli 2A | Embryonic carcinoma | + |
| 129 ES | Embryonic stem cell | − |
| CL-S1 | BALB/c mammary pre-neoplastic | +/− |
| | alveolar nodules | |
| CMT-93 | Rectal carcinoma | − |

Certain mouse 5T4-reactive antibodies are available, including mouse mAb B3F1 (Catalog #ab254168; Abcam, Waltham, MA, USA) and those in Table 4 reported Southgate, 2010). This antibody exhibits strong binding to 5T4 in ELISA, FACS, and Western blot assays and can be radiolabeled and used for targeting the 5T4-expressing mouse tumor cell line EMT6.

TABLE 4

| Antibody | Subclass | Epitope |
|---|---|---|
| B5C9 | IgG₁ | Proximal LRR |
| P1C9 | IgG_{2b} | Proximal LRR |
| P1H10 | IgG_{2b} | Proximal LRR |
| B1C3 | IgG₂ₐ | Distal LRR |
| B3F1 | IgG₂ₐ | Distal LRR |

Experimental plan: A exemplary experimental plan includes conjugation of a 5T4 targeting agent, such as antibody B3F1 with the chelator DOTA, following by radiolabeling with ¹¹¹In or ²²⁵Ac. Specific activity, efficiency of labeling, and stability of the radiolabeled antibody can then be determined as indicated above in Example 1.

An in vitro cell killing assay can be performed with the radiolabeled 5T4 targeting agent, such as ²²⁵Ac radiolabeled B3F1 antibody. EMT6 cells can be used as a positive control for cells that express 5T4 and may be exposed to a dilution series of ²²⁵Ac-labeled DOTA-B3F1 and unlabeled DOTA-B3F1 for 1 hour. Cell viability can be measured using an XTT assay as described hereinabove. If desired, a cell line that does not express 5T4 such as LL/2 cells (see Table 2, source—Woods, 2002) can be used as a negative control.

Table 3 shows a Fluorescent Activated Cell Sorting (FACS) analysis of the 9A7 antibody, presented in Woods (2002), against a panel of murine cell lines, wherein 105 cells of each line were stained with 9A7. The last column in Table 3 indicates the relative reactivity of 9A7 against the panel of cell lines, wherein the mammary cell line EMT6 is highly reactive and the lung carcinoma cell line LL/2 is non-reactive.

Biodistribution experiments: An ¹¹¹In labeled 5T4 targeting agent, such as B3F1 antibody, may be used in a first round of biodistribution experiments performed with tumor-free BALB/c mice to evaluate any binding of the antibody to normal tissues and to calculate absorbed dose of radiation to organs. A second round of biodistribution experiments can be performed using BALB/c mice bearing EMT6 tumors to evaluate specific targeting of antibody to the 5T4-expressing tumor and to calculate absorbed dose of radiation to the tumor and to other organs.

Following biodistribution experiments, tumor-bearing mice can be treated with escalating single doses of ²²⁵Ac-labeled 5T4 targeting agent, such as ²²⁵Ac-DOTA-B3F1, to establish the maximum tolerated dose (MTD) of the agent. The range of doses may, for example, be from 50 nCi to 400 nCi. Tolerability of the agent can be determined through measurements of body weight, behavior, and blood chemistry/counts.

Once the MTD has been established, experiments with variations in dosing frequency and combinations may be undertaken. Single MTD dose can be compared to fractionated dosing where the same total amount of radiation is administered across multiple doses, such as 2-4 doses. Combinations such as with immune checkpoint inhibitors may also be undertaken. Existing data (Barnes, 2018) suggests that an anti-CD137 antibody may be more effective than an anti-PD-1 as an immune checkpoint therapy.

### Example 3: Xenograft Mouse Model

Xenograft mouse models may be utilized to determine if a therapeutic targeting agent has an effect on human-derived cancerous cells. However, unless the targeting agent cross-reacts with the mouse target, it primarily only provides information about the cell-killing ability of the agent on the xenograft cells and may not provide information regarding on-target but off-tumor effects.

Exemplary human xenograft lines with varying levels of 5T4 expression are documented in Table 5 (source—Harper, 2017) and in Shi, 2019 which reports ZV0508 binding from highest to lowest for cell lines MDA-MB-468, DU145, BxPC-3, Lovo, HepG2 and Ramos. Although the mouse and human 5T4 protein have a high degree of similarity, the published antibodies targeting this protein do not cross-react with mouse and human 5T4. Therefore, while use of an anti-human 5T4 antibody in a xenograft model will only demonstrate anti-tumor reactivity, it can still provide important information, including the amount of radiation delivered to the tumor.

TABLE 5

| Cell Line | Cancer | 5T4/Cell |
|---|---|---|
| MDA-MB-361 | Breast | ~65,900 |
| MDA-MB-468 | Breast | ~41,800 |
| HCC1937 | Breast | ~34,000 |
| DU 145 | Prostate | ~30,400 |
| Calu 6 | Lung | ~18,000 |
| NCI-H1975 | Lung | ~15,800 |
| NCI-N87 | Gastric | ~4,400 |
| Capan-1 | Pancreatic | ~3,800 |
| DMS 114 | Lung | <100 |

There are numerous human 5T4 targeting therapies that have been developed for cancers. The original description of an anti-5T4 antibody sequence was provided by Hole & Stern (Hole, 1988). An antibody for use as an 5T4 targeting agent according to the presently disclosed invention, such as in preclinical studies, may be produced using the sequence provided by Hole & Stern. Alternatively, other 5T4 antibodies have been identified and include those used in the products by Medimmune/AstraZeneca (MEDI0641; Harper, 2017), Aptevo Therapeutics/Alligator Bioscience (ALG.APV-527), Biotecnol/Chiome Bioscience (Tb535), Guangdong Zhongsheng Pharmaceuticals (H6-DM5; Wang, 2018), and Zova Biotherapeutics (ZV0508; Shi, 2019). Additional antibodies that may be used, which are bispecific or available as antibody drug conjugates, are listed in Table 6 and provide additional possible 5T4 targeting agents, i.e., the 5T4 specific binding portions thereof.

The Medimmune/Astrazeneca antibody is of particular interest for use as an antibody radio-conjugate in methods of the presently disclosed invention due to the engineering of a cysteine, which can be used for site-specific conjugation of a chelator such as DOTA and chelation with a radioisotope, for example, as described in U.S. Provisional Patent Application No. 63/042,651 filed Dec. 9, 2019 entitled “Compositions and methods for preparation of site-specific radioconjugates,” and in U.S. Pat. No. 11,000,604—each of which is incorporated by reference herein. The present invention provides that any antibodies, such as those described herein and of any type (full-length, IgG, Fab fragment, single chain, scFv, nanobody, etc.) that have existing thiol groups available for labeling (on cysteines available for reaction under the conditions used) may be labeled by such methods, for example, using the PODS-DOTA linker of in U.S. Pat. No. 11,000,604, and that any such antibodies may be modified to include one or more free cysteines for such labeling.

TABLE 6

| Company Name | | | |
|---|---|---|---|
| (Originator) | Partners | Product Name | Agent |
| Active Biotech AB | NeoTX | naptumomab estafenatox | Antibody (Fab) - |
| | Therapeutics | (ABR-217620, second | staphylococcal |
| | Ltd. | generation) | enterotoxin A fusion |
| | | | protein |
| Aptevo Therapeutics | Alligator | ALG.APV-527 | Antibody (dual targeting) |
| Inc. | Bioscience AB | | |
| Asana BioSciences | Mersana | ASN004 | Antibody-drug conjugate; |
| LLC | | | scFvFc structure |
| Biotecnol Ltd. | Chiome | Tb535 | Antibody (triabody) |
| | Bioscience | | |
| Ambrx Inc. | Pfizer Inc. | Anti-5T4 ADC | Antibody-drug conjugate |
| Byondis | Previous name: | Anti-5T4 SYD1875 | Antibody-drug conjugate |
| | Synthon | | |
| Genmab | Abbvie | GEN1044 (DuoBody- | Bispecific mAb |
| | | CD3 × 5T4) | |
| Guangdong | — | H6-DM4 | Antibody-drug conjugate |
| Zhongsheng | | | |
| Pharmaceutical Co | | | |
| Medimmune | AstraZeneca | MEDI0641 | Antibody-drug conjugate |
| Oxford Biomedica | — | H8 | Antibody |
| Pfizer Inc. | Oxford | PF-06263507/ | Antibody-drug conjugate |
| | Biomedica | A1mcMMAF | |
| Zova Biotherapeutics | Neoantigen | ZV0508 | Antibody-drug conjugate |
| | Therapeutics | | |
| Macrogenics | — | 5T4 × CD137 TRIDENT | Bispecific mAb |
| Macrogenics | — | 5T4 × CD3 DART ® | Bispecific mAb |
| Amgen | — | 5T4-CD3 Bispecific | Bispecific mAb |

An experimental plan very similar to that described in Example 2 for the syngeneic mouse model can be carried out. Briefly, the anti-human 5T4 antibody can be radiolabeled, purified, and specific activity measured. In vitro cell killing experiments can be carried out, such as with any of the 5T4-expressing cell lines described in Table 7 or listed above.

Biodistribution experiments in tumor bearing mice as well as MTD studies can be carried out, such as described in Example 2. Once the MTD has been established, experiments with variations in dosing frequency and combinations can be undertaken. Single MTD dose can be compared to fractionated dosing where the same total amount of radiation is administered across multiple doses, such as 2-4 doses. Efficacy studies with the determined MTD, or other lower doses given in fractions, can also be carried out as described herein. Combination studies such as with immune checkpoint inhibitors and CD47 blockades, may also be undertaken.

TABLE 7

| Cancer | Cell lines |
|---|---|
| Breast cancer | MDAMB435 cells that overexpress |
| | 5T4 (Boghaert, 2008) |
| | MDAMB468 (Sapra, 2013; Shi, 2019) |
| | MDAMB361DYT2 (Sapra, 2013) |
| | MDAMB361(Harper, 2017) |
| Gastric cancer | N87 (Harper, 2017; Boghaert, |
| | 2008; Smith, 2018) |
| | HGC-27 (Wang, 2018) |
| | NSCLC: H157 (Boghaert, 2008) |
| | H1975 (Sapra, 2013) |
| Bronchioloalveolar cancer | PC14PE6 (Boghaert, 2008) |
| (orthotopic) | |
| Prostate cancer | DU145 (Harper, 2017; Shi, 2019) |
| Colon cancer | HT-29 (Wang, 2018) |
| | DLD-1 (Wang, 2018) |
| | HCT-15 (Wang, 2018) |
| | Lovo (Shi, 2019) |
| Pancreatic cancer | PANC-1 (Wang, 2018) |
| | BX-PC3 (Shi, 2019; Wang, 2018) |
| Ovarian cancer | SKOV-3 (Owens, 2018) |
| Cervical epidermoid cancer | A431 (Smith, 2018) |

### Example 4: Patient Derived Xenograft Model

Patient derived xenografts (PDX) are very similar to the xenograft mouse models except the cancerous cells are directly derived from a patient tumor and have undergone no or very few passages in cell culture, making them more representative of human tumors.

PDX models have been utilized in agents targeting 5T4, as described by Kerk and colleagues (Kerk, 2017), Sapra and colleagues (Sapra, 2013), and Wang and colleagues (Wang, 2018).

Therefore, a PDX model may also be utilized for both in vitro and in vivo preclinical testing of a radiolabeled 5T4 targeting agent such as an antibody, using an experimental plan similar to that described for syngeneic and xenograft mouse models.

### Example 5: Exemplary PARPi Administration and Dosing Regimes

(A) Olaparib (Lynparza®)—Normal and Reduced Dosing Regimens

Olaparib is sold by AstraZeneca under the brand name Lynparza®. Lynparza® is sold in tablet form at 100 mg and 150 mg. The dosage is 300 mg taken orally twice daily for a daily total of 600 mg. Dosing continues until disease progression or unacceptable toxicity. This dosing regimen is referred to herein as the “normal” human dosing regimen for Lynparza®, regardless of the disorder treated. Any dosing regimen having a shorter duration (e.g., 21 days) or involving the administration of less Lynparza® (e.g., 300 mg/day) is referred to herein as a “reduced” human dosing regimen. Examples of reduced human dosing regimens include the following: (i) 550 mg/day; (ii) 500 mg/day; (iii) 450 mg/day; (iv) 400 mg/day; (v) 350 mg/day; (vi) 300 mg/day; (vii) 250 mg/day; (viii) 200 mg/day; (ix) 150 mg/day; (x) 100 mg/day; or (xi) mg/day.

(B) Niraparib (Zejula®)—Normal and Reduced Dosing Regimens

Niraparib is sold by Tesaro under the brand name Zejula®. Zejula® is sold in capsule form at 100 mg. The dosage is 300 mg taken orally once daily. Dosing continues until disease progression or unacceptable adverse reaction. This dosing regimen is referred to herein as the “normal” human dosing regimen for Zejula®, regardless of the disorder treated. Any dosing regimen having a shorter duration (e.g., 21 days) or involving the administration of less Zejula® (e.g., 150 mg/day) is referred to herein as a “reduced” human dosing regimen. Examples of reduced human dosing regimens include the following: (i) 250 mg/day; (ii) 200 mg/day; (iii) 150 mg/day; (iv) 100 mg/day; or (v) 50 mg/day.

(C) Rucaparib (Rubraca®)—Normal and Reduced Dosing Regimens

Rucaparib is sold by Clovis Oncology, Inc. under the brand name Rubraca™. Rubraca™ is sold in tablet form at 200 mg and 300 mg. The dosage is 600 mg taken orally twice daily for a daily total of 1,200 mg. Dosing continues until disease progression or unacceptable toxicity. This dosing regimen is referred to herein as the “normal” human dosing regimen for Rubraca™, regardless of the disorder treated. Any dosing regimen having a shorter duration (e.g., 21 days) or involving the administration of less Rubraca™ (e.g., 600 mg/day) is referred to herein as a “reduced” human dosing regimen. Examples of reduced human dosing regimens include the following: (i) 1,150 mg/day; (ii) 1,100 mg/day; (iii) 1,050 mg/day; (iv) 1,000 mg/day; (v) 950 mg/day; (vi) 900 mg/day; (vii) 850 mg/day; (viii) 800 mg/day; (ix) 750 mg/day; (x) 700 mg/day; (xi) 650 mg/day; (xii) 600 mg/day; (xiii) 550 mg/day; (xiv) 500 mg/day; (xv) 450 mg/day; (xvi) 400 mg/day; (xvii) 350 mg/day; (xviii) 300 mg/day; (xix) 250 mg/day; (xx) 200 mg/day; (xxi) 150 mg/day; or (xxii) 100 mg/day.

(D)—Talazoparib (Talzenna™)—Normal and Reduced Dosing Regimens

Talazoparib is sold by Pfizer Labs under the brand name Talzenna™. Talzenna™ is sold in capsule form at 1 mg. The dosage is 1 mg taken orally. Dosing continues until disease progression or unacceptable toxicity. This dosing regimen is referred to herein as the “normal” human dosing regimen for Talzenna™, regardless of the disorder treated. Any dosing regimen having a shorter duration (e.g., 21 days) or involving the administration of less Talzenna™ (e.g., mg/day) is referred to herein as a “reduced” human dosing regimen. Examples of reduced human dosing regimens include the following: (i) 0.9 mg/day; (ii) 0.8 mg/day; (iii) 0.7 mg/day; (iv) 0.6 mg/day; (v) 0.5 mg/day; (vi) 0.4 mg/day; (vii) 0.3 mg/day; (viii) 0.2 mg/day; or (ix) 0.1 mg/day.

### Example 6: Dosing Regimens for 5T4 Targeting Agent and PARPi

A human patient may be treated according to the following regimen. One of olaparib, niraparib, rucaparib or talazoparib (PARPi) is orally administered according to one of the dosing regimens listed in Example 5, accompanied by intravenous administration of a radiolabeled 5T4 targeting agent as detailed herein in either single or fractional administration. For example, the dosing regimens include, by way of example: (a) the PARPi and the 5T4 targeting agent administered concurrently, wherein (i) each is administered beginning on the same day, (ii) the 5T4 targeting agent is administered in a single dose or fractionated doses not less than one week apart, and (iii) the PARPi is administered daily or twice daily (as appropriate), and for a duration equal to or exceeding that of the 5T4 targeting agent administration; or (b) the PARPi and 5T4 targeting agent are administered concurrently, wherein (i) the PARPi administration precedes 5T4 targeting agent administration by at least one week, (ii) the 5T4 targeting agent is administered in a single dose or fractionated doses not less than one week apart, and (iii) the PARPi is administered daily or twice daily (as appropriate), and for a duration equal to or exceeding that of the 5T4 targeting agent administration.

Without limitation, the following aspects of the invention are also provided:

Aspect 1. A method for treating a solid cancer, such as any of those disclosed herein, or acute lymphoblastic leukemia (ALL), such as Ph+ ALL, in a mammalian subject, such as a human patient, the method including: administering to the subject a therapeutically effective amount of a radiolabeled 5T4 targeting agent such as any of the 5T4 targeting agents disclosed herein.

Aspect 2. The method according to aspect 1, wherein the cancer is a breast cancer, triple-negative breast cancer (TNBC), metastatic breast cancer, gastric cancer, bladder cancer, cervical cancer, endometrial cancer, skin cancer, stomach cancer, testicular cancer, esophageal cancer, bronchioloalveolar cancer, prostate cancer, metastatic prostate cancer, colorectal cancer, ovarian cancer, cervical epidermoid cancer, pancreatic cancer, lung cancer, non-small cell lunger cancer (NSCLC), renal cancer, head and neck cancer, mesothelioma, or any combination thereof.

Aspect 3. The method according to any preceding aspect, wherein the cancer is colorectal cancer, gastric cancer, ovarian cancer, non-small cell lung carcinoma, head and neck squamous cell cancer, pancreatic cancer, renal cancer, or any combination thereof.

Aspect 4. The method according to any preceding aspect, wherein the cancer is a cancer such as a 5T4-positive tumor.

Aspect 5. The method according to any preceding aspect, wherein the radiolabeled 5T4 targeting agent includes a radiolabel selected from ¹³¹I, ¹²⁵I, ¹²³I, ⁹⁰Y, ¹⁷⁷Lu, ¹⁸⁶Re, ¹⁸⁸Re, ⁸⁹Sr, ¹⁵³Sm, ³²P, ²²⁵Ac, ²¹³Bi, ²¹³Po, ²¹¹At, ²¹²Bi, ²¹³Bi, ²²³Ra, ²²⁷Th, ¹⁴⁹Tb, ¹³⁷Cs, ²¹²Pb or ¹⁰³Pd, or a combination thereof.

Aspect 6. The method according to any preceding aspect, wherein the radiolabeled 5T4 targeting agent includes a radiolabel selected from ¹³¹I, ⁹⁰Y, ¹⁷⁷Lu, ²²⁵Ac, ²¹³Bi, ²¹¹At, ²¹³Bi, ²²⁷Th, ²¹²Pb, or a combination thereof.

Aspect 7. The method according to any preceding aspect, wherein the radiolabeled 5T4 targeting agent includes an antibody against 5T4 (a 5T4-specific antibody).

Aspect 8. The method according to any preceding aspect, wherein the 5T4 targeting agent includes the monoclonal antibody or 5T4-binding monoclonal antibody component of MEDI0641, ALG.APV-527, Tb535, H6-DM5, Naptumomab estafenatox, or ZV0508.

Aspect 9. The method according to any one of aspects 1 to 6, wherein the 5T4 targeting agent is a peptide or small molecule.

Aspect 10. The method according to any preceding aspect, wherein the effective amount of the radiolabeled 5T4 targeting agent is a maximum tolerated dose.

Aspect 11. The method according to any preceding aspect, wherein the radiolabeled 5T4 targeting agent is ²²⁵Ac-, ¹⁷⁷Lu-, or ¹³¹I-labeled.

Aspect 12. The method according to any preceding aspect, wherein the therapeutically effective amount of the radiolabeled 5T4 targeting agent includes a single dose that delivers less than 2 Gy, or less than 8 Gy, such as doses of 2 Gy to 8 Gy, to the subject.

Aspect 13. The method according to any preceding aspect, wherein the radiolabeled 5T4 targeting agent is ²²⁵Ac-labeled, and the effective amount of the ²²⁵Ac-labeled targeting agent includes a dose of 0.1 to 50 μCi/kg body weight of the subject, or 0.2 to 20 μCi/kg body weight of the subject, or 0.5 to 10 μCi/kg subject body weight.

Aspect 14. The method according to any preceding aspect, wherein the radiolabeled 5T4 targeting agent is a full length antibody against 5T4 that is ²²⁵Ac-labeled, and the effective of the ²²⁵Ac-labeled 5T4 targeting agent includes less than 5 μCi/kg body weight of the subject, such as 0.1 to 5 μCi/kg body weight of the subject.

Aspect 15. The method according to any one of aspects 1 to 11, wherein the radiolabeled 5T4 targeting agent is an antibody fragment, such as a minibody or nanobody against 5T4 that is ²²⁵Ac-labeled, and the effective of the ²²⁵Ac-labeled 5T4 targeting agent includes greater than 5 μCi/kg body weight of the subject, such as 5 to 20 μCi/kg body weight of the subject.

Aspect 16. The method according to any one of aspects 1 to 11, wherein the radiolabeled 5T4 targeting agent is ²²⁵Ac-labeled, and the effective amount of the ²²⁵Ac-labeled targeting agent includes 2 μCi to 2 mCi, or 2 μCi to 250 μCi, or 75 μCi to 400 μCi.

Aspect 17. The method according to any one of aspects 1 to 11, wherein the radioisotope labeled 5T4 targeting agent is ¹⁷⁷Lu-labeled and the effective amount of the 5T4 targeting agent includes a dose of less than 1000 μCi/kg body weight of the subject, such as a dose of 1 to 900 μCi/kg body weight of the subject, or 5 to 250 μCi/kg body weight of the subject or 50 to 450 μCi/kg body weight.

Aspect 18. The method according to any one of aspects 1 to 11, wherein the radioisotope labeled 5T4 targeting agent is ¹⁷⁷Lu-labeled, and the effective amount of the ¹⁷⁷Lu-labeled 5T4 targeting agent includes a dose of 10 mCi to below 30 mCi, or from at least 100 μCi to below 3 mCi, or from 3 mCi to below 30 mCi.

Aspect 19. The method according to any one of aspects 1 to 11, wherein the radiolabeled 5T4 targeting agent is ¹³¹I-labeled, and the effective amount of the ¹³¹I-labeled 5T4 targeting agent includes a dose of less than 1200 mCi, such as a dose of 25 to 1200 mCi, or 100 to 400 mCi, or 300 to 600 mCi, or 500 to 1000 mCi.

Aspect 20. The method according to any one of aspects 1 to 11, wherein the radiolabeled 5T4 targeting agent is ¹³¹I-labeled, and the effective amount of the ¹³¹I-labeled 5T4 targeting agent includes a dose of less than 200 mCi, such as a dose of 1 to 200 mCi, or 25 to 175 mCi, or 50 to 150 mCi.

Aspect 21. The method according to any preceding aspect, wherein the effective amount of the 5T4 targeting agent includes a protein dose of less than 3 mg/kg body weight of the subject, such as from 0.001 mg/kg patient weight to 3.0 mg/kg patient weight, or from 0.005 mg/kg patient weight to 2.0 mg/kg patient weight, or from 0.01 mg/kg patient weight to 1 mg/kg patient weight, or from 0.1 mg/kg patient weight to 0.6 mg/kg patient weight, or 0.3 mg/kg patient weight, or 0.4 mg/kg patient weight, or 0.5 mg/kg patient weight, or 0.6 mg/kg patient weight.

Aspect 22. The method according to any preceding aspect, wherein the 5T4 targeting agent is administered according to a dosing schedule selected from the group consisting of once every 7, 10, 12, 14, 20, 24, 28, 36, and 42 days throughout a treatment period, wherein the treatment period includes at least two doses.

Aspect 23. The method according to any preceding aspect, further including administering to the subject a therapeutically effective amount of an immune checkpoint therapy, a CD47 blockade, a chemotherapeutic agent, a DNA damage response inhibitor (DDRi), or any combination thereof.

Aspect 24. The method according to aspect 23, wherein the DDRi includes a poly(ADP-ribose) polymerase inhibitor (PARPi), an ataxia telangiectasia mutated inhibitor (ATMi), an ataxia telangiectasia mutated and Rad-3 related inhibitor (ATRi), or a Wee1 inhibitor.

Aspect 25. The method according to aspect 23, wherein the chemotherapeutic agent administered 7, 8, 9, 10, and/or 11 days after the 5T4 targeting agent.

Aspect 26. The method according to aspect 23, wherein the immune checkpoint therapy includes an antibody against CTLA-4, PD-1, TIM-3, VISTA, BTLA, LAG-3, TIGIT, CD28, OX40, GITR, CD137, CD40, CD40L, CD27, HVEM, PD-L1, PD-L2, PD-L3, PD-L4, CD80, CD86, CD137-L, GITR-L, CD226, B7-H3, B7-H4, BTLA, TIGIT, GALS, KIR, 2B4, CD160, CGEN-15049, or a combination thereof.

Aspect 27. The method according to aspect 26, wherein the immune checkpoint therapy includes an antibody against PD-1, PD-L1, PD-L2, CTLA-4, CD137, or a combination thereof.

Aspect 28. The method according to aspect 24, wherein the PARPi includes one or more of olaparib, niraparib, rucaparib and talazoparib.

Aspect 29. The method according to aspect 24, wherein the ATMi includes one or more of KU-55933, KU-59403, wortmannin, CP466722, or KU-60019.

Aspect 30. The method according to aspect 24, wherein the ATRi includes one or more of Schisandrin B, NU6027, NVP-BEA235, VE-821, VE-822, AZ20, or AZD6738.

Aspect 31. The method according to aspect 21, wherein the Wee1 inhibitor includes AZD-1775 (i.e., adavosertib).

Aspect 32. The method according to aspect 23, wherein the 5T4 targeting agent is administered at least one week before the immune checkpoint therapy, CD47 blockade, and/or the DDRi; or wherein the immune checkpoint therapy and/or the DDRi is administered at least one week before the 5T4 targeting agent.

Aspect 33. The method according to aspect 23, wherein the 5T4 targeting agent is administered with one of the immune checkpoint therapy, CD47 blockade or the DDRi, and another of the immune checkpoint therapy, CD47 blockade or the DDRi is administered either before or after the 5T4 targeting agent.

Aspect 34. The method according to aspect 23, wherein the 5T4 targeting agent is administered simultaneously with the immune checkpoint therapy and/or the DDRi.

Aspect 35. The method according to any preceding aspect, wherein the 5T4 targeting agent is a multi-specific antibody, wherein the multi-specific antibody includes: a first target recognition component which specifically binds to an epitope of 5T4, and a second target recognition component which specifically binds to a different epitope of 5T4 than the first target recognition component, or an epitope of a different antigen.

Aspect 36. A method for treating a proliferative disease or disorder, the method including: diagnosing the subject with 5T4-positive cells; and if the subject has 5T4-positive cells, administering to the subject a therapeutically effective amount of an 5T4 targeting agent according to any of the methods of aspects 1 to 35.

Aspect 37. The method according to aspect 36, wherein the diagnosing includes obtaining a sample of blood or tissue from the subject; mounting the sample on a substrate; and detecting the presence or absence of 5T4 antigen using a diagnostic antibody, wherein the diagnostic antibody includes an antibody against 5T4 labeled with a radiolabel such as ³H, ¹⁴C, ³²P, ³⁵S, and ¹²⁵I, and/or labeled with a fluorescent or chemiluminescent compound, such as fluorescein isothiocyanate, rhodamine, or luciferin, and/or labeled with an enzyme, such as alkaline phosphatase, β-galactosidase, or horseradish peroxidase.

Aspect 38. The method according to aspect 36, wherein the diagnosing includes administering a 5T4 targeting agent to the subject, wherein the 5T4 targeting agent includes a radiolabel selected from the group including ¹⁸F, ¹¹C, ⁶⁸Ga, ⁶⁴Cu, ⁸⁹Zr, ¹²⁴I, ^{99m}Tc, or ¹¹¹In; waiting a time sufficient to allow the 5T4 targeting agent to accumulate at a tissue site; and then imaging the tissues with a non-invasive imaging technique to detect presence or absence of 5T4-positive cells.

Aspect 39. The method according to aspect 38, wherein the non-invasive imaging technique includes positron emission tomography (PET imaging) for ¹⁸F, ¹¹C, ⁶⁸Ga, ⁶⁴Cu, ⁸⁹Zr or ¹²⁴I labeled 5T4 targeting agents or single photon emission computed tomography (SPECT imaging) for ^{99m}Tc or ¹¹¹In labeled 5T4 targeting agents.

Aspect 40. A pharmaceutical composition for treating a cancer in a mammalian subject, such as a human patient, the composition comprising a therapeutically effective amount of a radionuclide labeled 5T4 targeting agent, such as any of those disclosed herein, wherein the cancer is a solid cancer, such as any of those disclosed herein, or a hematological cancer such as any of those disclosed herein such as acute lymphoblastic leukemia (ALL), such as Ph+ ALL.

Aspect 41. The pharmaceutical composition of aspect 40, wherein the cancer is a breast cancer, triple negative breast cancer, metastatic breast cancer, gastric cancer, bladder cancer, cervical cancer, endometrial cancer, skin cancer, stomach cancer, testicular cancer, esophageal cancer, bronchioloalveolar cancer, prostate cancer, metastatic prostate cancer, colorectal cancer, ovarian cancer, cervical epidermoid cancer, pancreatic cancer, lung cancer, NSCLC, renal cancer, head and neck cancer, or any combination thereof.

Aspect 42. The pharmaceutical composition of aspect 40 or 41, wherein the radionuclide is ¹³¹I, ¹²⁵I, ¹²³I, ⁹⁰Y, ¹⁷⁷Lu, ¹⁸⁶Re, ¹⁸⁸Re, ⁸⁹Sr, ¹⁵³Sm, ³²P, ²²⁵Ac, ²¹³Bi, ²¹³Po, ²¹¹At, ²¹²Bi, ²¹³Bi, ²²³Ra, ²²⁷Th, ¹⁴⁹Tb, ¹³⁷Cs, ²¹²Pb or ¹⁰³Pd.

Aspect 43. The pharmaceutical composition of any one of aspects 40-42, further comprising at least one pharmaceutically acceptable excipient.

Aspect 44. Any of the preceding aspects, wherein the solid cancer(s) recited is metastatic.

Solid tumor cancers referred to herein may be non-metastatic or metastatic. Thus, wherever in this disclosure an aspect of the invention relates to a solid cancer, related aspects directed to metastatic forms of the cancer as well as to non-metastatic forms of the cancer are disclosed.

While various specific aspects have been illustrated and described herein, it will be appreciated that various changes thereto may be made without departing from the spirit and scope of the invention. Moreover, features described in connection with one aspect of the invention may be embodied in and used in conjunction with other aspects of the invention, even if not explicitly exemplified in combination within.

### REFERENCES

Barnes, S., EMT-6 Syngeneic Breast Tumor Model A Powerful Tool for Immuno-Oncology Studies, (2018).
Bartkowiak, T. et al., 4-1BB Agonists: Multi-Potent Potentiators Of Tumor Immunity, Frontiers Oncol. 5:117; pp. 1-16 (2015).
Boghaert, E. et al. The oncofetal protein, 5T4, is a suitable target for antibody-guided anti-cancer chemotherapy with calicheamicin. Int. J. Oncol. 32, 221-234 (2008).
Borghaei, H. et al. Phase I Dose Escalation, Pharmacokinetic and Pharmacodynamic Study of Naptumomab Estafenatox Alone in Patients With Advanced Cancer and With Docetaxel in Patients With Advanced Non-Small-Cell Lung Cancer. J. Clin. Oncol. 27, 4116-4123 (2009).
Castro, F. V. et al. 5T4 oncofoetal antigen is expressed in high risk of relapse childhood pre-B acute lymphoblastic leukemia and is associated with a more invasive and chemotactic phenotype, Leukemia. July; 26(7): 1487-1498 (2012)
Cheng, J. D. et al. Individualized Patient Dosing in Phase I Clinical Trials: The Role of Escalation With Overdose Control in PNU-214936. J. Clin. Oncol. 22, 602-609 (2004).
Gelmon, et al., Olaparib in Patients with Recurrent High-Grade Serous or Poorly Differentiated Ovarian Carcinoma or Triple-Negative Breast Cancer: A Phase 2, Multicentre, Open-Label, Non-Randomized Study. Lancet Oncol. 12:852-61 (2011).
Harper, J. et al. Preclinical evaluation of MEDI0641, a pyrrolobenzodiazepine-conjugated antibody-drug conjugate targeting 5T4. Mol. Cancer Ther. 16, 1576-1587 (2017).
Harrop, R. et al. Vaccination of castration-resistant prostate cancer patients with TroVax (MVA-5T4) in combination with docetaxel: a randomized phase II trial. Cancer Immunol. Immunother. 62(9), 1511-1520 (2013).
Hole, N. & Stern, P. L. A 72 kD trophoblast glycoprotein defined by a monoclonal antibody. Br. J. Cancer 57, 239-246 (1988).
Kerk, S. A. et al. 5T4-Targeted Therapy Ablates Cancer Stem Cells and Prevents Recurrence of Head and Neck Squamous Cell Carcinoma. Clin. Cancer Res. 23, 2516-2527 (2017).
Kwon, B., Is CD137 Ligand (CD137L) Signaling a Fine Tuner of Immune Responses?, Immune Network. 15(3): 121-124 (2015).
Lamichhane, P., Amin, N., Agarwal, M. & Lamichhane, N. Checkpoint Inhibition: Will Combination with Radiotherapy and Nanoparticle-Mediated Delivery Improve Efficacy? Medicines 5, 114 (2018).
Owens, G. L. et al. Preclinical Assessment of CAR T-Cell Therapy Targeting the Tumor Antigen 5T4 in Ovarian Cancer. J. Immunother. 41, 130-140 (2018).
Sapra, P. et al. Long-term Tumor Regression Induced by an Antibody-Drug Conjugate That Targets 5T4, an Oncofetal Antigen Expressed on Tumor-Initiating Cells. Mol. Cancer Ther. 12, 38-47 (2013).
Shapiro, G. I. et al. First-in-human trial of an anti-5T4 antibody-monomethylauristatin conjugate, PF-06263507, in patients with advanced solid tumors. Invest. New Drugs 35, 315-323 (2017).
Sharma, S. K. et al. A rapid bead-based radioligand binding assay for the determination of target-binding fraction and quality control of radiopharmaceuticals. Nucl. Med. Biol. 71, 32-38 (2019).
Shaw, D. M. et al. A phase II study of a 5T4 oncofoetal antigen tumour-targeted superantigen (ABR-214936) therapy in patients with advanced renal cell carcinoma. Br. J. Cancer 96, 567-574 (2007).
Shi, B. et al. Antitumor activity of a 5T4 targeting antibody drug conjugate with a novel payload derived from MMAF via C-Lock linker. Cancer Med. 8, 1793-1805 (2019).
Smith, R. A., Zammit, D. J. & Reddy, S. P. Abstract B109: ASN004, a novel 5T4-targeted Dolaflexin ADC, causes complete and durable tumor regression in a variety of tumor xenograft models. in Therapeutic Agents: Biological 17, B109-B109 (American Association for Cancer Research, 2018).
Southall, P. J. et al. Immunohistological distribution of 5T4 antigen in normal and malignant tissues. Br. J. Cancer 61, 89-95 (1990).
Southgate, T. D. et al. CXCR4 Mediated Chemotaxis Is Regulated by 5T4 Oncofetal Glycoprotein in Mouse Embryonic Cells. PLoS One 5, e9982 (2010).
Stern, P. L. & Harrop, R. 5T4 oncofoetal antigen: an attractive target for immune intervention in cancer. Cancer Immunol. Immunother. 66, 415-426 (2017).
Twyman-Saint Victor, C. et al. Radiation and dual checkpoint blockade activate non-redundant immune mechanisms in cancer. Nature 520, 373-377 (2015).
Wang, R. et al. A novel 5T4-targeting antibody-drug conjugate H6-DM4 exhibits potent therapeutic efficacy in gastrointestinal tumor xenograft models. Am. J. Cancer Res. 8, 610-623 (2018).
Wei, H. et al., Dual Targeting of CD137 Co-Stimulatory and PD-1 Co-Inhibitory Molecules for Ovarian Cancer Immunotherapy, OncoImmunology 3; pp. 1-3 (2014).
Woods, A. M. et al. Characterization of the murine 5T4 oncofoetal antigen: a target for immunotherapy in cancer. Biochem. J. 366, 353-365 (2002).

1. A method for treating a cancer in a mammalian subject, the method comprising:
administering to the subject a therapeutically effective amount of a 5T4 targeting agent which is (i) radionuclide labeled Naptumomab estafenatox, or (ii) an antibody heavy chain comprising SEQ ID NO:20 bound to an antibody light chain comprising SEQ ID NO:15, wherein either or both of the heavy chain and the light chain are radionuclide labeled,
wherein the cancer is a solid cancer or acute lymphoblastic leukemia (ALL).

2. The method of claim 1, wherein the administering step comprising administering a composition comprising the 5T4 targeting agent in which a first portion of the 5T4 targeting agent is radionuclide labeled and the remaining portion is not radionuclide labeled.

3. The method of claim 1, wherein the 5T4 targeting agent is chemically conjugated to a chelator.

4. The method of claim 3, wherein the chelator comprises DOTA.

5. The method of claim 1, wherein the cancer is a 5T4-expressing cancer.

6. The method of claim 1, wherein the mammalian subject is human.

7. The method of claim 1, wherein, wherein the 5T4 targeting agent is radionuclide labeled with ¹³¹I, ¹²⁵I, ¹²³I, ⁹⁰Y, ¹⁷⁷Lu, ¹⁸⁶Re, ¹⁸⁸Re, ⁸⁹Sr, ¹⁵³Sm, ³²P, ²²⁵Ac, ²¹³Bi, ²¹³Po, ²¹¹At, ²¹²Bi, ²¹³Bi, ²²³Ra, ²²⁷Th, ¹⁴⁹Tb, ¹³⁷Cs, or ²¹²Pb.

8. The method of claim 7, wherein the 5T4 targeting agent is ²²⁵Ac-labeled, and the effective amount of the ²²⁵Ac-labeled 5T4 targeting agent comprises a dose of 0.1 to 50 μCi/kg body weight of the subject, or 0.1 to 5 μCi/kg body weight of the subject, or 5 to 20 μCi/kg subject body weight.

9. The method of claim 7, wherein the 5T4 targeting agent is ²²⁵Ac-labeled, and the effective amount of the ²²⁵Ac-labeled 5T4 targeting agent comprises a dose of 2 μCi to 2 mCi, or 2 μCi to 250 Xi, or 75 μCi to 400 μCi.

10. The method of claim 7, wherein the 5T4 targeting agent is ¹³¹I-labeled and the effective amount of the 5T4 targeting agent comprises a dose of less than 200 mCi, such as a dose of 1 to 200 mCi, or 25 to 175 mCi, or 50 to 150 mCi.

11. The method of claim 1, wherein the effective amount of the 5T4 targeting agent comprises a protein dose of less than 3 mg/kg body weight of the subject, such as from 0.001 mg/kg patient weight to 3.0 mg/kg patient weight, or from 0.005 mg/kg patient weight to 2.0 mg/kg patient weight, or from 0.01 mg/kg patient weight to 1 mg/kg patient weight, or from 0.1 mg/kg patient weight to 0.6 mg/kg patient weight, or 0.3 mg/kg patient weight, or 0.4 mg/kg patient weight, or 0.5 mg/kg patient weight, or 0.6 mg/kg patient weight.

12-40. (canceled)

41. A method for treating a cancer in a mammalian subject, the method comprising:
administering to the subject a therapeutically effective amount of a radionuclide labeled targeting agent,
wherein the cancer is a solid cancer or acute lymphoblastic leukemia (ALL).

42. The method of claim 41, wherein the solid cancer is a breast cancer, gastric cancer, bladder cancer, cervical cancer, endometrial cancer, skin cancer, stomach cancer, testicular cancer, esophageal cancer, bronchioloalveolar cancer, prostate cancer, colorectal cancer, ovarian cancer, cervical epidermoid cancer, pancreatic cancer, lung cancer, renal cancer, head and neck cancer, mesothelioma, or any combination thereof.

43. The method of claim 41, wherein the solid cancer is colorectal cancer, gastric cancer, ovarian cancer, non-small cell lung carcinoma, head and neck squamous cell cancer, pancreatic cancer, renal cancer, or any combination thereof.

44. The method of claim 41, wherein the solid cancer comprises a 5T4-positive tumor.

45. The method of claim 41, wherein the radionuclide labeled 5T4 targeting agent comprises a radiolabel selected from ¹³¹I, ¹²⁵I, ¹²³I, ⁹⁰Y, ¹⁷⁷Lu, ¹⁸⁶Re, ¹⁸⁸Re, ⁸⁹Sr, ¹⁵³Sm, ³²P, ²²⁵Ac, ²¹³Bi, ²¹³Po, ²¹¹At, ²¹²Bi, ²¹³Bi, ²²³Ra, ²²⁷Th, ¹⁴⁹Tb, ¹³⁷Cs, ²¹²Pb or ¹⁰³Pd, or any combination thereof.

46. The method of claim 41, wherein the radionuclide labeled 5T4 targeting agent comprises a radiolabel selected from ²²⁵Ac, ¹⁷⁷Lu, ¹³¹I, ⁹⁰Y, ²¹³Bi, ²¹¹At, ²¹³Bi, ²²⁷Th, ²¹²Pb, or any combination thereof.

47. The method of claim 41, wherein the radionuclide labeled 5T4 targeting agent comprises a humanized antibody against 5T4.

48. The method of claim 41, wherein the radionuclide labeled 5T4 targeting agent comprises Naptumomab estafenatox, Naptumomab estafenatox modified to include one or more new cysteines, Naptumomab estafenatox modified to include one or more extra amino acids in the light chain portion and/or the heavy chain portion which amino acids may include a cysteine or a lysine, the antibody portion of Naptumomab estafenatox, MEDI0641, at least the 5T4-binding portions of ALG.APV-527, at least the 5T4-binding portion of Tb535, mAb H6, or ZV05.

49-54. (canceled)

55. The method of claim 41, further comprising:
administering to the subject a therapeutically effective amount of an immune checkpoint therapy, a CD47 blockade, a DNA damage response inhibitor (DDRi), a chemotherapeutic agent, or any combination thereof.

56. The method of claim 55, comprising administering a therapeutically effective amount of an immune checkpoint therapy comprising an antibody against PD-1, PD-L1, PD-L2, CTLA-4, CD137, or any combination thereof.

57-73. (canceled)


SEQUENCE LISTING (33 sequences)

SEQ ID NO: 1 - protein, 420 aa, Homo sapiens
MPGGCSRGPAAGDGRLRLARLALVLLGWVSSSSPTSSASSFSSSAPFLASAVSAQPPLPDQCPALCECSEAARTVKCVNRNLTEVPTDLPAYVRNLFLTGNQLAVLPAGAFARRPPLAELAALNLSGSRLDEVRAGAFEHLPSLRQLDLSHNPLADLSPFAFSGSNASVSAPSPLVELILNHIVPPEDERQNRSFEGMVVAALLAGRALQGLRRLELASNHFLYLPRDVLAQLPSLRHLDLSNNSLVSLTYVSFRNLTHLESLHLEDNALKVLHNGTLAELQGLPHIRVFLDNNPWVCDCHMADMVTWLKETEVVQGKDRLTCAYPEKMRNRVLLELNSADLDCDPILPPSLQTSYVFLGIVLALIGAIFLLVLYLNRKGIKKWMHNIRDACRDHMEGYHYRYEINADPRLTNLSSNSDV

SEQ ID NO: 2 - protein, 493 aa, Artificial Sequence
  scFv
EVQLVESGGGLVQPGGSLRLSCKASGYSFTGYYMHWVRQAPGKGLEWVSRINPNNGVTLYNQKFKDRFTISRDNSKNTLYLQMNSLRAEDTAVYYCARSTMITNYVMDYWGQGTLVTVSSGGGGSGGGGSGGGGSGGGGSGGGGSGGGGSDIQMTQSPSSLSASVGDRVTITCKASQSVSNDVAWYQQKPGKAPKLLIYYTSSRYAGVPSRFSGSGSGTDFTLTISSLQPEDFATYYCQQDYNSPPTFGGGTKLEIKASTCEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 142 aa, Artificial sequence
  Synthetic
MDWTWRFLFVVAAATGVQSQVQLVQSGGGLVQPGGSLRLSCAASGFTFSSYEMNWVRQAPGKGLEWVSYISSSGSTIYYADSVKGRFTISRDNAKNSLYLQMNSLRAEDTAVYYCAREMQFGWELLGAFDIWGQGTMVTVSS

SEQ ID NO: 4 - protein, 127 aa, Artificial sequence
  Synthetic
MDMRVPAQLLGLLLLWLSGARCEIVLTQSPGTLSLSPGERATLSCRASQSVSSSYLAWYQQKPGQAPRLLIYGASSRATGIPDRFSGSGSGTDFTLTISRLEPEDFAVYYCQQYGSSFGQGTKLEIK

SEQ ID NO: 5 - protein, 472 aa, Artificial sequence
  Synthetic
MDWTWRFLFVVAAATGVQSQVQLVQSGGGLVQPGGSLRLSCAASGFTFSSYEMNWVRQAPGKGLEWVSYISSSGSTIYYADSVKGRFTISRDNAKNSLYLQMNSLRAEDTAVYYCAREMQFGWELLGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 234 aa, Artificial sequence
  Synthetic
MDMRVPAQLLGLLLLWLSGARCEIVLTQSPGTLSLSPGERATLSCRASQSVSSSYLAWYQQKPGQAPRLLIYGASSRATGIPDRFSGSGSGTDFTLTISRLEPEDFAVYYCQQYGSSFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 233 aa, Staphylococcus sp
SEKSEEINEKDLRKKSELQRNALSNLRQIYYYNEKAITENKESDDQFLENTLLFKGFFTGHPWYNDLLVDLGSKDATNKYKGKKVDLYGAYYGYQCAGGTPNKTACMYGGVTLHDNNRLTEEKKVPINLWIDGKQTTVPIDKVKTSKKEVTVQELDLQARHYLHGKFGLYNSDSFGGKVQRGLIVFHSSEGSTVSYDLFDAQGQYPDTLLRIYRDNKTINSENLHIDLYLYTT

SEQ ID NO: 8 - protein, 233 aa, Staphylococcus sp
SEKSEEINEKDLRKKSELQGTALGNLKQIYYYNEKAKTENKESHDQFLQHTILFKGFFTDHSWYNDLLVDFDSKDIVDKYKGKKVDLYGAYYGYQCAGGTPNKTACMYGGVTLHDNNRLTEEKKVPINLWLDGKQNTVPLETVKTNKKNVTVQELDLQARRYLQEKYNLYNSDVFDGKVQRGLIVFHTSTEPSVNYDLFGAQGQYSNTLLRIYRDNKTINSENMHIDIYLYTS

SEQ ID NO: 9 - protein, 233 aa, Artificial Sequence
  Mutated Protein
SEKSEEINEKDLRKKSELQGTALGNLKQIYYYNSKAITSSEKSADQFLTNTLLFKGFFTGHPWYNDLLVDLGSTAATSEYEGSSVDLYGAYYGYQCAGGTPNKTACMYGGVTLHDNNRLTEEKKVPINLWIDGKQTTVPIDKVKTSKKEVTVQELDLQARHYLHGKFGLYNSDSFGGKVQRGLIVFHSSEGSTVSYDLFDAQGQYPDTLLRIYRDNTTISSTSLSISLYLYTT

SEQ ID NO: 10 - protein, 233 aa, Artificial Sequence
  Mutated Protein
SEKSEEINEKDLRKKSELQGTALGNLKQIYYYNEKAKTENKESHDQFLQHTILFKGFFTDHSWYNDLLVDFDSKDIVDKYKGKKVDLYGAYYGYQCAGGTPNKTACMYGGVTLHDNNRLTEEKKVPINLWLDGKQNTVPLETVKTNKKNVTVQELDLQARRYLQEKYNLYNSDVFDGKVQRGLIVFHTSTEPSVNYDLFGAQGQYSNTLLRIYRDNKTINSENMHIAIYLYTS

SEQ ID NO: 11 - protein, 679 aa, Artificial Sequence
  Conjugated Protein
  MISC_FEATURE (460)..(679): Light Chain
QVQLQQPGAELVRPGASVKLSCKASGYTFTNYWINWVKQRPGQGLEWIGNIYPSYIYTNYNQEFKDKVTLTVDESSSTAYMQLSSPTSEDSAVYYCTRSPYGYDEYGLDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDSGGPSEKSEEINEKDLRKKSELQGTALGNLKQIYYYNEKAKTENKESHDQFLQHTILFKGFFTDHSWYNDLLVDFDSKDIVDKYKGKKVDLYGAYYGYQCAGGTPNKTACMYGGVTLHDNNRLTEEKKVPINLWLDGKQNTVPLETVKTNKKNVTVQELDLQARRYLQEKYNLYNSDVFDGKVQRGLIVFHTSTEPSVNYDLFGAQGQYSNTLLRIYRDNKTINSENMHIDIYLYTSDIVMTQSPSSLTVTAGEKVTMNCKSSQSLLNSRNQKNYLTWYQQKPGQPPKLLIYWASTRESGVPDRFTGSGSGTDFTLTISSVQAEDLAVYYCQNDYVYPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNES

SEQ ID NO: 12 - protein, 672 aa, Artificial Sequence
  Mutated and Conjugated Protein
  MISC_FEATURE (459)..(672): Light Chain
EVQLQQSGPDLVKPGASVKISCKASGYSFTGYYMHWVKQSHGKSLEWIGRINPNNGVTLYNQKFKDKAILTVDKSSTTAYMELRSLTSEDSAVYYCARSTMITNYVMDYWGQVTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDSGGPSEKSEEINEKDLRKKSELQGTALGNLKQIYYYNEKAKTENKESHDQFLQHTILFKGFFTDHSWYNDLLVDFDSKDIVDKYKGKKVDLYGAYYGYQCAGGTPNKTACMYGGVTLHDNNRLTEEKKVPINLWLDGKQNTVPLETVKTNKKNVTVQELDLQARRYLQEKYNLYNSDVFDGKVQRGLIVFHTSTEPSVNYDLFGAQGQYSNTLLRIYRDNKTINSENMHIAIYLYTSSIVMTQTPTSLLVSAGDRVTITCKASQSVSNDVAWYQQKPGQSPKLLISYTSSRYAGVPDRFSGSGSGTDFTLTISSVQAEDLAVYFCQQDYNSPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNES

SEQ ID NO: 13 - protein, 672 aa, Artificial Sequence
  Mutated and Conjugated Protein
  MISC_FEATURE (459)..(672): Light Chain
EVQLQQSGPDLVKPGASVKISCKASGYSFTGYYMHWVKQSPGKGLEWIGRINPNNGVTLYNQKFKDKATLTVDKSSTTAYMELRSLTSEDSAVYYCARSTMITNYVMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDSGGPSEKSEEINEKDLRKKSELQGTALGNLKQIYYYNSKAITSSEKSADQFLTNTLLFKGFFTGHPWYNDLLVDLGSTAATSEYEGSSVDLYGAYYGYQCAGGTPNKTACMYGGVTLHDNNRLTEEKKVPINLWIDGKQTTVPIDKVKTSKKEVTVQELDLQARHYLHGKFGLYNSDSFGGKVQRGLIVFHSSEGSTVSYDLFDAQGQYPDTLLRIYRDNTTISSTSLSISLYLYTTSIVMTQTPTSLLVSAGDRVTITCKASQSVSNDVAWYQQKPGQSPKLLISYTSSRYAGVPDRFSGSGYGTDFTLTISSVQAEDAAVYFCQQDYNSPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNES

SEQ ID NO: 14 - protein, 458 aa, Artificial Sequence
  Synthetic polypeptide
EVQLQQSGPDLVKPGASVKISCKASGYSFTGYYMHWVKQSPGKGLEWIGRINPNNGVTLYNQKFKDKATLTVDKSSTTAYMELRSLTSEDSAVYYCARSTMITNYVMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDSGGPSEKSEEINEKDLRKKSELQGTALGNLKQIYYYNSKAITSSEKSADQFLTNTLLFKGFFTGHPWYNDLLVDLGSTAATSEYEGSSVDLYGAYYGYQCAGGTPNKTACMYGGVTLHDNNRLTEEKKVPINLWIDGKQTTVPIDKVKTSKKEVTVQELDLQARHYLHGKFGLYNSDSFGGKVQRGLIVFHSSEGSTVSYDLFDAQGQYPDTLLRIYRDNTTISSTSLSISLYLYTT

SEQ ID NO: 15 - protein, 214 aa, Artificial Sequence
  Synthetic polypeptide
SIVMTQTPTSLLVSAGDRVTITCKASQSVSNDVAWYQQKPGQSPKLLISYTSSRYAGVPDRFSGSGYGTDFTLTISSVQAEDAAVYFCQQDYNSPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNES

SEQ ID NO: 16 - protein, 233 aa, Artificial Sequence
  Mutated Protein
SEKSEEINEKDLRKKSELQGTALGNLKQIYYYNEKAITENKESDDQFLENTLLFKGFFTGHPWYNDLLVDLGSKDATNKYKGKKVDLYGAYYGYQCAGGTPNKTACMYGGVTLHDNNRLTEEKKVPINLWIDGKQTTVPIDKVKTSKKEVTVQELDLQARHYLHGKFGLYNSDSFGGKVQRGLIVFHSSEGSTVSYDLFDAQGQYPDTLLRIYRDNKTINSENLHIALYLYTT

SEQ ID NO: 17 - protein, 215 aa, Artificial Sequence
  Synthetic polypeptide
SIVMTQTPTSLLVSAGDRVTITCKASQSVSNDVAWYQQKPGQSPKLLISYTSSRYAGVPDRFSGSGYGTDFTLTISSVQAEDAAVYFCQQDYNSPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNESK

SEQ ID NO: 18 - protein, 215 aa, Artificial Sequence
  Synthetic polypeptide
SIVMTQTPTSLLVSAGDRVTITCKASQSVSNDVAWYQQKPGQSPKLLISYTSSRYAGVPDRFSGSGYGTDFTLTISSVQAEDAAVYFCQQDYNSPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNESC

SEQ ID NO: 19 - protein, 216 aa, Artificial Sequence
  Synthetic polypeptide
SIVMTQTPTSLLVSAGDRVTITCKASQSVSNDVAWYQQKPGQSPKLLISYTSSRYAGVPDRFSGSGYGTDFTLTISSVQAEDAAVYFCQQDYNSPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNESKC

SEQ ID NO: 20 - protein, 222 aa, Artificial Sequence
  Synthetic polypeptide
EVQLQQSGPDLVKPGASVKISCKASGYSFTGYYMHWVKQSPGKGLEWIGRINPNNGVTLYNQKFKDKATLTVDKSSTTAYMELRSLTSEDSAVYYCARSTMITNYVMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDS

SEQ ID NO: 21 - protein, 225 aa, Artificial Sequence
  Synthetic polypeptide
EVQLQQSGPDLVKPGASVKISCKASGYSFTGYYMHWVKQSPGKGLEWIGRINPNNGVTLYNQKFKDKATLTVDKSSTTAYMELRSLTSEDSAVYYCARSTMITNYVMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDSGGP

SEQ ID NO: 22 - protein, 223 aa, Artificial Sequence
  Synthetic polypeptide
EVQLQQSGPDLVKPGASVKISCKASGYSFTGYYMHWVKQSPGKGLEWIGRINPNNGVTLYNQKFKDKATLTVDKSSTTAYMELRSLTSEDSAVYYCARSTMITNYVMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDSK

SEQ ID NO: 23 - protein, 226 aa, Artificial Sequence
  Synthetic polypeptide
EVQLQQSGPDLVKPGASVKISCKASGYSFTGYYMHWVKQSPGKGLEWIGRINPNNGVTLYNQKFKDKATLTVDKSSTTAYMELRSLTSEDSAVYYCARSTMITNYVMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDSGGPK

SEQ ID NO: 24 - protein, 223 aa, Artificial Sequence
  Synthetic polypeptide
EVQLQQSGPDLVKPGASVKISCKASGYSFTGYYMHWVKQSPGKGLEWIGRINPNNGVTLYNQKFKDKATLTVDKSSTTAYMELRSLTSEDSAVYYCARSTMITNYVMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDSC

SEQ ID NO: 25 - protein, 226 aa, Artificial Sequence
  Synthetic polypeptide
EVQLQQSGPDLVKPGASVKISCKASGYSFTGYYMHWVKQSPGKGLEWIGRINPNNGVTLYNQKFKDKATLTVDKSSTTAYMELRSLTSEDSAVYYCARSTMITNYVMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDSGGPC

SEQ ID NO: 26 - protein, 224 aa, Artificial Sequence
  Synthetic polypeptide
EVQLQQSGPDLVKPGASVKISCKASGYSFTGYYMHWVKQSPGKGLEWIGRINPNNGVTLYNQKFKDKATLTVDKSSTTAYMELRSLTSEDSAVYYCARSTMITNYVMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDSKC

SEQ ID NO: 27 - protein, 227 aa, Artificial Sequence
  Synthetic polypeptide
EVQLQQSGPDLVKPGASVKISCKASGYSFTGYYMHWVKQSPGKGLEWIGRINPNNGVTLYNQKFKDKATLTVDKSSTTAYMELRSLTSEDSAVYYCARSTMITNYVMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDSGGPKC

SEQ ID NO: 28 - protein, 673 aa, Artificial Sequence
  Mutated and Conjugated Protein
  MISC_FEATURE (459)..(672): Light Chain
EVQLQQSGPDLVKPGASVKISCKASGYSFTGYYMHWVKQSPGKGLEWIGRINPNNGVTLYNQKFKDKATLTVDKSSTTAYMELRSLTSEDSAVYYCARSTMITNYVMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDSGGPSEKSEEINEKDLRKKSELQGTALGNLKQIYYYNSKAITSSEKSADQFLTNTLLFKGFFTGHPWYNDLLVDLGSTAATSEYEGSSVDLYGAYYGYQCAGGTPNKTACMYGGVTLHDNNRLTEEKKVPINLWIDGKQTTVPIDKVKTSKKEVTVQELDLQARHYLHGKFGLYNSDSFGGKVQRGLIVFHSSEGSTVSYDLFDAQGQYPDTLLRIYRDNTTISSTSLSISLYLYTTSIVMTQTPTSLLVSAGDRVTITCKASQSVSNDVAWYQQKPGQSPKLLISYTSSRYAGVPDRFSGSGYGTDFTLTISSVQAEDAAVYFCQQDYNSPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNESK

SEQ ID NO: 29 - protein, 459 aa, Artificial Sequence
  Synthetic polypeptide
EVQLQQSGPDLVKPGASVKISCKASGYSFTGYYMHWVKQSPGKGLEWIGRINPNNGVTLYNQKFKDKATLTVDKSSTTAYMELRSLTSEDSAVYYCARSTMITNYVMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDSGGPSEKSEEINEKDLRKKSELQGTALGNLKQIYYYNSKAITSSEKSADQFLTNTLLFKGFFTGHPWYNDLLVDLGSTAATSEYEGSSVDLYGAYYGYQCAGGTPNKTACMYGGVTLHDNNRLTEEKKVPINLWIDGKQTTVPIDKVKTSKKEVTVQELDLQARHYLHGKFGLYNSDSFGGKVQRGLIVFHSSEGSTVSYDLFDAQGQYPDTLLRIYRDNTTISSTSLSISLYLYTTK

SEQ ID NO: 30 - protein, 673 aa, Artificial Sequence
  Mutated and Conjugated Protein
  MISC_FEATURE (459)..(672): Light Chain
EVQLQQSGPDLVKPGASVKISCKASGYSFTGYYMHWVKQSPGKGLEWIGRINPNNGVTLYNQKFKDKATLTVDKSSTTAYMELRSLTSEDSAVYYCARSTMITNYVMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDSGGPSEKSEEINEKDLRKKSELQGTALGNLKQIYYYNSKAITSSEKSADQFLTNTLLFKGFFTGHPWYNDLLVDLGSTAATSEYEGSSVDLYGAYYGYQCAGGTPNKTACMYGGVTLHDNNRLTEEKKVPINLWIDGKQTTVPIDKVKTSKKEVTVQELDLQARHYLHGKFGLYNSDSFGGKVQRGLIVFHSSEGSTVSYDLFDAQGQYPDTLLRIYRDNTTISSTSLSISLYLYTTSIVMTQTPTSLLVSAGDRVTITCKASQSVSNDVAWYQQKPGQSPKLLISYTSSRYAGVPDRFSGSGYGTDFTLTISSVQAEDAAVYFCQQDYNSPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNESC

SEQ ID NO: 31 - protein, 459 aa, Artificial Sequence
  Synthetic polypeptide
EVQLQQSGPDLVKPGASVKISCKASGYSFTGYYMHWVKQSPGKGLEWIGRINPNNGVTLYNQKFKDKATLTVDKSSTTAYMELRSLTSEDSAVYYCARSTMITNYVMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDSGGPSEKSEEINEKDLRKKSELQGTALGNLKQIYYYNSKAITSSEKSADQFLTNTLLFKGFFTGHPWYNDLLVDLGSTAATSEYEGSSVDLYGAYYGYQCAGGTPNKTACMYGGVTLHDNNRLTEEKKVPINLWIDGKQTTVPIDKVKTSKKEVTVQELDLQARHYLHGKFGLYNSDSFGGKVQRGLIVFHSSEGSTVSYDLFDAQGQYPDTLLRIYRDNTTISSTSLSISLYLYTTC

SEQ ID NO: 32 - protein, 674 aa, Artificial Sequence
  Mutated and Conjugated Protein
  MISC_FEATURE (459)..(672): Light Chain
EVQLQQSGPDLVKPGASVKISCKASGYSFTGYYMHWVKQSPGKGLEWIGRINPNNGVTLYNQKFKDKATLTVDKSSTTAYMELRSLTSEDSAVYYCARSTMITNYVMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDSGGPSEKSEEINEKDLRKKSELQGTALGNLKQIYYYNSKAITSSEKSADQFLTNTLLFKGFFTGHPWYNDLLVDLGSTAATSEYEGSSVDLYGAYYGYQCAGGTPNKTACMYGGVTLHDNNRLTEEKKVPINLWIDGKQTTVPIDKVKTSKKEVTVQELDLQARHYLHGKFGLYNSDSFGGKVQRGLIVFHSSEGSTVSYDLFDAQGQYPDTLLRIYRDNTTISSTSLSISLYLYTTSIVMTQTPTSLLVSAGDRVTITCKASQSVSNDVAWYQQKPGQSPKLLISYTSSRYAGVPDRFSGSGYGTDFTLTISSVQAEDAAVYFCQQDYNSPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNESKC

SEQ ID NO: 33 - protein, 460 aa, Artificial Sequence
  Synthetic polypeptide
EVQLQQSGPDLVKPGASVKISCKASGYSFTGYYMHWVKQSPGKGLEWIGRINPNNGVTLYNQKFKDKATLTVDKSSTTAYMELRSLTSEDSAVYYCARSTMITNYVMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDSGGPSEKSEEINEKDLRKKSELQGTALGNLKQIYYYNSKAITSSEKSADQFLTNTLLFKGFFTGHPWYNDLLVDLGSTAATSEYEGSSVDLYGAYYGYQCAGGTPNKTACMYGGVTLHDNNRLTEEKKVPINLWIDGKQTTVPIDKVKTSKKEVTVQELDLQARHYLHGKFGLYNSDSFGGKVQRGLIVFHSSEGSTVSYDLFDAQGQYPDTLLRIYRDNTTISSTSLSISLYLYTTKC